(19) United States
(12) Patent Application Publication
(10) Pub. No.: US 2008/0311140 A1
(43) Pub. Date: Dec. 18, 2008

(54) Antigen specific immunosuppression by dendritic cell therapy

(75) Inventors: Brendan Lee, Houston, TX (US); Michael Seiler, Chicago, IL (US); Vincenzo Cerullo, Helsinki (FI)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Baylor College of Medicine, Houston, TX (US)

(21) Appl. No.: 12/156,179

(22) Filed: May 29, 2008

Related U.S. Application Data
(60) Provisional application No. 60/932,156, filed on May 29, 2007.

(51) Int. Cl. A61K 39/00; C12N 5/10; A61P 37/00

(52) U.S. Cl. 424/184.1; 435/372; 424/93.21

(57) ABSTRACT

The invention includes genetically modified dendritic cells expressing at least two immunosuppressive molecules. The genetically modified dendritic cells have the ability to induce tolerance. Enhanced tolerogenicity is useful for prolonging survival of a foreign transplant and for treatment of autoimmune diseases.

### CROSS-REFERENCE TO RELATED APPLICATIONS

This application claims priority under 35 U.S.C. § 119(e) to U.S. Provisional Patent Application No. 60/932,156, filed May 29, 2007, which is incorporated by reference herein in its entirety.

### STATEMENT REGARDING FEDERALLY SPONSORED RESEARCH OR DEVELOPMENT

This invention was made, in part, using funds obtained from the U.S. Government (National Institutes of Health Grant No. NIDDK DK56787), and the U.S. Government may therefore have certain rights in this invention.

### BACKGROUND OF THE INVENTION

The ability of T cells to recognize an antigen is dependent on the association of the antigen with either major histocompatibility complex (MHC) I or MHC II proteins. For example, cytotoxic T cells respond to an antigen that is presented in association with MHC-I proteins. Thus, a cytotoxic T cell that should kill virus-infected cell will not kill that cell if the cell does not also express the appropriate MHC-I protein. Helper T cells recognize antigen presented on MHC-II proteins. Helper T cell activity depends, in general, on the recognition of the antigen in complex with MHC-II proteins on antigen presenting cells. The requirement for recognition of an antigen in association with a MHC protein is essential for adaptive immunity, i.e., stimulation of an antibody response or cell mediated response to an antigen. MHC-I proteins are found on the surface of virtually all nucleated cells. MHC-II proteins are expressed on the surface of antigen presenting cells including macrophages, B cells, and dendritic cells (DCs) of the spleen and lymph nodes, as well as Langerhans cells of the skin, and mesenchymal stromal cells of the bone marrow.

A crucial step in mounting an adaptive immune response in mammals is the activation of CD4+ helper T-cells that recognize MHC-II restricted exogenous antigens. These antigens are captured and processed in the cellular endosomal pathway in antigen presenting cells, such as dendritic cells. In the endosome and lysosome, the antigen is processed into small antigenic peptides that are complexed onto MHC-II to form an antigen-MHC-II complex. This complex is expressed on the cell surface, which expression induces the activation of CD4+ T cells.

Other crucial events in the induction of an effective immune response in mammals involve the activation of CD8+ T-cells and B cells. CD8+ cells are activated when the desired protein is routed through the cell in such a manner so as to be presented on the cell surface as a processed protein, which is complexed with MHC-I proteins. B cells can interact with antigen via their surface immunoglobulins (IgM and IgD) without the need for MHC proteins. However, activation of CD4+ helper T-cells stimulates all arms of the immune system. Upon activation, CD4+ T-cells produce multiple cytokines, to tailor the immune response to the stimulus. These interleukins help activate the other arms of the immune system. For example, helper T cells produce interleukin-4 (IL-4) and interleukin-5 (IL-5), which help B cells produce antibodies; interleukin-2 (IL-2), which activates CD4+ and CD8+ T-cells; and gamma interferon, which activates macrophages.

Since helper T-cells that recognize MHC-II restricted antigens play a central role in the activation and clonal expansion of cytotoxic T-cells, macrophages, natural killer cells and B cells, the initial event of activating the helper T cells in response to an antigen is crucial for the induction of an effective immune response directed against that antigen.

In addition to the critical roles that T cells play in the immune response, DCs are equally important. DCs are professional antigen-presenting cells having a key regulatory role in the maintenance of tolerance to self-antigens and in the activation of innate and adaptive immunity against foreign antigens (Banchereau et al., 1998, Nature 392:245-52; Steinman et al., 2003, Annu. Rev. Immunol. 21:685-711). When DCs encounter pro-inflammatory stimuli such as microbial products, the maturation process of the cell is initiated by up-regulating cell surface expressed antigenic peptide-loaded MHC molecules, co-stimulatory molecules, and the secretion of pro-inflammatory cytokines. Following maturation and homing to local lymph nodes, DCs establish contact with T cells by forming an immunological synapse, where the T cell receptor (TCR) and co-stimulatory molecules congregate in a central area surrounded by adhesion molecules (Dustin et al., 2000, Nat. Immunol. 1:23-9). Once activated, CD8+ T cells can autonomously proliferate for several generations and acquire cytotoxic function without further antigenic stimulation (Kaech et al., 2001, Nat. Immunol. 2:415-22; van Stipdonk et al., 2001, Nat. Immunol. 2:423-9).

Autoimmune disorders are characterized by the loss of tolerance against self-antigens, activation of lymphocytes reactive against “self” antigens (autoantigens), and pathological damage in target organs. Normally, autoimmunity can also be prevented by peripheral tolerance, which is a process presumably involving a series of multi-step interactions between APCs, in particular DCs, and effector T cells.

A role for DCs in central tolerance induction was initially demonstrated in the context of self-tolerance within the thymus, in which DCs stimulate the deletion of self-reactive T cells. Both myeloid and lymphoid DC populations have been reported to be able to induce peripheral, antigen-specific unresponsiveness in various experimental models, or have been implicated as having a role in self-tolerance. Mechanisms whereby DCs accomplish this goal include selective activation of Th2 subsets, induction of regulatory T cells, induction of T cell anergy, and induction of T cell apoptosis. The acceptance of this concept is facilitated by the identification of DC subsets, whose functions are affected (and perhaps dictated) by micro-environmental factors, in particular cytokines, IL-10, TGF-β, prostaglandin E2, and corticosteroids.

Other molecules also influence DC function. For example, the chimeric fusion protein cytotoxic T lymphocyte antigen 4 (CTLA4)-Ig can render DCs tolerogenic. Fas ligand (CD95 L) that is expressed on lymphoid or myeloid DCs and tumor necrosis factor-related apoptosis-inducing ligand (TRAIL) that is expressed on human CD11c⁺ blood DCs may regulate or eliminate T cells responding to antigens presented by DCs. Thus, genetically engineered DCs expressing immuno-modulatory molecules, such as viral IL-10 (vIL-10), TGF-β, Fas ligand, or CTLA4Ig have been developed. For instance delivery of IL-10 into mature DCs has been found to promote tolerogenicity (Lu et al., 1999, J. Leukoc. Biol. 66:293-296) and delivery of cytotoxic CTLA4Ig into mature DCs has also been shown to promote tolerogenicity and survival of these DCs in allogeneic recipients (Lu et al., 1999, Gene Ther. 6:554-563). In addition, delivery of TGF-β into DCs has been found to prevent the reduction of DCs generally seen with adenovirus infection and also increase the numbers and prolong the survival of the infected DCs in the spleen of a host to whom the DCs have been administered (Lee et al., 1998, Transplantation 66:1810-1817).

The mammalian immune system plays a central role in protecting individuals from infectious agents and preventing tumor growth. However, the same immune system can produce undesirable effects such as the rejection of cell, tissue and organ transplants from unrelated donors. The immune system does not distinguish beneficial intruders, such as a transplanted tissue, from those that are harmful, and thus the immune system rejects transplanted tissues or organs. Rejection of transplanted organs is generally mediated by alloreactive T cells present in the host which recognize donor alloantigens or xenoantigens.

The transplantation of cells, tissues, and organs between genetically disparate individuals invariably results in the risk of graft rejection. Nearly all cells express products of the major histocompatibility complex, MHC class I molecules. Further, antigen presenting cells can be induced to express MHC class II molecules carrying foreign tissue antigens when exposed to inflammatory cytokines. Additional immunogenic molecules include those derived from minor histocompatibility antigens such as Y chromosome antigens recognized by female recipients. Rejection of allografts is mediated primarily by T cells of both the CD4 and CD8 subclasses (Rosenberg et al., 1992, Annu. Rev. Immunol. 10:333). Alloreactive CD4+ T cells produce cytokines that exacerbate the cytolytic CD8 response to alloantigen. Within these subclasses, competing subpopulations of cells develop after antigen stimulation and they are characterized by the cytokines they produce. Th1 cells, which produce IL-2 and IFN-γ, are primarily involved in allograft rejection (Mossmann et al., 1989, Annu. Rev. Immunol. 7:145). Th2 cells, which produce IL-4, IL-5 and IL-10, can down-regulate Th1 responses through IL-10 (Fiorentino et., 1989, J. Exp. Med. 170:2081). Indeed, much effort has been expended to divert undesirable Th1 responses toward the Th2 pathway. Undesirable alloreactive T cell responses in patients (allograft rejection, graft-versus-host disease) are typically handled with immunosuppressive drugs such as prednisone, azathioprine, and cyclosporine A. Unfortunately, these drugs generally need to be maintained for the life of the patient and they have a multitude of dangerous side effects including generalized immunosuppression. A much better approach than pan immunosuppression is to induce specific or localized suppression to donor cell alloantigens, leaving the remaining immune system intact.

Unwanted CD4+ immune responses leading to B cell activation and the production of antibodies is a major problem not only in autoimmune disease, but also in situations of protein therapy delivered either exogenously or produced endogenously as per after gene therapy. Examples of the former are the generation of inhibitory antibodies to factor VIII protein infusion for the treatment of hemophilia and the production of antibodies against anti-TNF-α antibody treatments. In fact, this is a predictable and general response to therapies that involve delivery of antigen not previously present during the immunological maturation of the recipient. These unwanted immune response limit efficacy of the intervention and are associated with unwanted toxicity.

Unwanted antibody responses to protein therapies or to self antigens are important clinical problems (Steinman et al., 2002 PNAS 99: 351-358). This is particularly relevant to the X-linked disorder of Hemophilia A caused by the absence of functional clotting factor VIII (FVIII); where approximately 25% of patients receiving recombinant protein therapy make inhibitory antibodies to the FVIII molecule (Addiego et al., 1993 Lancet 342: 462-464; Lusher et al., 1993 Transfusion 33: 791-793; Lusher et al., 1993 N Engl J. Med. 328: 453-459; Oldenburg et al., 2002 Haemophilia 2: 23-29. Consistent with this inhibitor formation, gene transfer strategies using various different vectors to treat both hemophilia A and hemophilia B in pre-clinical animal models have been plagued by the induction of anti-transgene immunity (Herzog et al., 2002 Hum Gene Ther. 13: 1281-1291; Brown et al., 2004 J Thromb Haemost. 2: 111-118; McCormack et al., 2006 J Thromb Haemost. 4: 1218-1225. Therefore, developing a method to control or suppress detrimental immunity in an antigen-specific fashion is integral to the long term success of therapies requiring repeated protein administration, as well as to the endogenous production of potential therapeutic neo-antigens after gene replacement.

While modification of DCs may be an attractive approach to the therapy of foreign graft rejection and autoimmune disorders as well as cell therapy to suppress anticipated, unwanted immune responses to prolong gene therapy, there are potential problems associated with such an approach. Tolerogenicity may be enhanced in a host by the administration of immature DCs which are hyporesponsive. However, infection of DCs with an adenoviral vector alone stimulates maturation of DCs and enhances the immunostimulatory capacity of DCs, and hence, their ability to engage T cells (Rea et al., 1999 J. Virol. 73:10245-10253). In addition, it has been shown that infection of DCs with an adenovirus expressing eGFP enhanced costimulatory molecule expression and induction of CTL responses of both TGF-β and IL-4 in a dose dependent manner.

Therefore, there is a need for a method for producing DCs which do not readily stimulate immunity when introduced into a host. In addition, there is a need for a method of enhancing tolerogenicity in a host (such as autoimmune disease) using DCs that which exert tolerogenic properties. Furthermore, there is a need for a method of producing tolerogenic DCs comprising a vector wherein the genetically modified DCs maintain their tolerogenicity in the presence of the vector. Finally, there is a need for such DCs to exert their tolerogenic or immunsuppressive actions in an antigen specific fashion without general suppression on the immune system.

### BRIEF SUMMARY OF THE INVENTION

The present invention relates to novel antigen presenting cells, preferably, dendritic cells (DCs), capable of inducing tolerance.

In one embodiment, the DCs are genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof.

In another embodiment, the DC can further comprise an antigen having at least one epitope.

In one aspect, the DC comprises an antigen expressed by an expression vector. In some instances, the antigen is delivered directly as a pulse of a protein. In other instances, the antigen is delivered directly as a mixture of proteins either purified or from cell/tissue lysates.

In a further aspect, the antigen is associated with a disease wherein the disease is selected from the group consisting of an infectious disease, a cancer and an autoimmune disease.

In yet a further aspect, the antigen is associated with a therapeutic treatment.

The present invention also includes a method of inducing immune tolerance in a mammal.

In one embodiment, the method comprises administering a DC to a mammal in need thereof, wherein the DC is genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β, (TGFβ), and any combination thereof.

In one aspect, the DC can further comprise an antigen having at least one epitope. In some instances, the antigen is expressed by an expression vector. In other instances, the antigen is delivered directly as a pulse of a protein. In yet other instances, the antigen is delivered directly as a mixture of proteins either purified or from cell/tissue lysates. The antigen can be associated with a disease, wherein the disease is selected from the group consisting of an infectious disease, a cancer and an autoimmune disease or a therapeutic treatment. The antigen can also be associated with an autoimmune disease.

The invention also encompasses a method of treating a transplant recipient to reduce in the recipient an immune response against the transplant.

In one embodiment, the method comprises administering to a transplant recipient, a DC genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof, in an amount effective to reduce an immune response against the transplant.

In one aspect, the transplant is selected from the group consisting of a biocompatible lattice, a donor tissue, an organ, a cell, a nucleic acid, a protein, and any combination thereof.

In another aspect, the DC further comprises an antigen having at least one epitope, wherein the antigen is associated with the transplant. In some instances, the antigen is expressed by an expression vector. In other instances, the antigen is delivered directly as a pulse of a protein. In yet other instances, the antigen is delivered directly as a mixture of proteins either purified or from cell/tissue lysates.

In another aspect, the DC is administered to the transplant recipient to treat rejection of the transplant by the recipient. In another aspect, the DC is administered to the transplant recipient in combination with an immunosuppressive agent.

In some aspects, the DCs are administered to the recipient prior to the transplant. In other aspects, the DCs are administered to the recipient concurrently with the transplant. In yet other aspects, the DCs are administered as part of the transplant. In still another aspect, the DCs are administered to the recipient subsequent to the transplantation of the transplant.

The invention also includes a method of enhancing the expression of a protein in a mammal. The method comprises administering a dendritic cell genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof, into said mammal thereby enhancing expression of said protein.

In one embodiment, the DC comprises an antigen having at least one epitope. In another embodiment, the antigen is expressed in said cell by an expression vector. In yet another embodiment, the antigen is delivered directly to the DC as a pulse of a protein. In another embodiment, the antigen is delivered directly to the DC as a mixture of proteins that are purified or are from cell/tissue lysates. Preferably, the antigen is associated with the protein that is targeted for enhanced expression.

In one embodiment, the protein is expressed in the mammal as a result of gene therapy. In another embodiment, the protein expressed in the mammal is a therapeutic protein. A therapeutic protein includes, but is not limited, to a hormone, a monoclonal antibody, an enzyme, a cytokine, a toxin, a fusion protein, and the like.

In one embodiment, the protein that is targeted for enhanced expression includes, but is not limited to FVIII, insulin, thrombopoietin (TPO), erythropoietin (EPO), interferon-β (INF-β), INF-α, GM-CSF, tissue plasminogen activator, myelin basic protein (MBP), AXO, and the like.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting obstacles to clinical gene therapy highlighting the problem of unwanted immune responses to a therapeutic protein.

FIG. 2, comprising FIGS. 2A through D, is a series of images demonstrating the generation of tolerogenic DC by helper-dependent adenoviral gene transfer. FIG. 2A depicts images of fluorescence microscopy of GFP expression of transduced of DC using calcium-phosphate precipitation (CaPi) mediated helper dependent adenovirus. FIG. 2A is a schematic of a construct of a helper-dependent adenovirus (HD-Ad) expressing the cytokines TGFβ and IL-10 (HDAd_{Tol}). FIG. 2C is an image of restriction digest confirmation of the TGF-β/IL-10 transgene-containing pΔ28E4 helper-dependent vector backbone. FIG. 2D is a chart depicting IL-6 and TNF-α secretion after HD-Ad5TGF-β/IL-10:CaPi transduction of DC.

FIG. 3, comprising FIGS. 3A through 3H, is a series of charts depicting the characteristics of DCₜₒₗ. FIGS. 3A through 3C is a series of charts demonstrating that HDAd_{Tol} significantly reduced DC expression of the maturation markers CD40 and CD86 (FIG. 3A), as well as reduced secretion of TNFα (FIG. 3C) and IL-6 (FIG. 3B). FIG. 3D is a chart depicting DCₜₒₗ reduce the frequency of CD4⁺ T cells in vitro.

FIGS. 3E and 3F are charts demonstrating that DCₜₒₗ increases the frequency of CD4⁺ T cells in apoptosis in vitro. FIGS. 3G and 3H are charts demonstrating DCₜₒₗ decreases the frequency of bystander CD4⁺ T cells, but does not inhibit proliferation of responders in vitro.

FIG. 4, comprising FIGS. 4A through 4C, is a series of images demonstrating that DCₜₒₗ induce T cell apoptosis in vitro. FIG. 4A is a chart depicting percentage of TCRtg⁺, CD4⁺, and TCRtg⁻, CD4⁺ T cells expressing the apoptotic marker annexin V after 24 hours in syngeneic co-culture with wild type BALB/cJ DC after the indicated treatment, loaded with the D011.10 TCRtg antigen OVA or irrelevant hAAT. FIG. 4B is a chart depicting the mean percentage of apoptotic (Annexin V⁺) TCRtg⁺, CD4⁺ and TCRtg⁻, CD4⁺T cells after 24 hour co-culture with OVA loaded, syngeneic BALB/cJ DCs. FIG. 4C is an image depicting mean percentage of apoptotic (Annexin V⁺) TCRtg⁺, CD4⁺ and TCRtg⁻, CD4⁺ T cells after 24 hour co-culture with hAAT loaded, syngeneic BALB/cJ DCs.

FIG. 5 is a chart demonstrating that DCₜₒₗ increase the frequency of antigen-specific regulatory T cells in vitro.

FIG. 6, comprising 6A and 6B, is a series of charts demonstrating that DCₜₒₗ suppresses the antigen-specific immunization response in vivo. FIG. 6A is a schematic of the experimental model where syngeneic DCₜₒₗ were loaded with either hAAT or human albumin and adoptively transferred into recipient C3H/HeJ mice two times, one week apart. FIG. 6B is a chart demonstrating the anti-albumin antibody titer after adoptive transfer with either DCₜₒₗ loaded with hAAT (DCₜₒₗ-hAAT) or albumin (DCₜₒₗ-alb).

FIG. 7, comprising FIGS. 7A and 7B, is a series of chart demonstrating that DCₜₒₗ adoptive transfer prolongs FVIII gene therapy in vivo. FIG. 7A is a schematic of the experimental model where syngeneic DCₜₒₗ, or control DC_{HD-AdGFP} transduced with a GFP expressing HD vector or untreated DC (mock) were loaded with recombinant human FVIII and adoptively transferred into recipient FVIII knockout mice two times, one week apart. FIG. 7B is a chart depicting the percentage of mice expressing detectible FVIII over time.

FIG. 8, comprising FIGS. 8A through 8B, is a series of charts demonstrating that DCₜₒₗ suppresses the anti-FVIII immune response, but not the anti-adenovirus response in vivo. FIG. 8A is a chart depicting total Anti-FVIII IgG antibody titer twenty-four weeks after systemic gene transfer. FIG. 8B is a chart demonstrating that the ability of recipient-mouse serum to neutralize adenovirus in vitro was measured twenty-four weeks post systemic gene transfer.

FIG. 9, comprising FIGS. 9A through 9B, is a series of charts demonstrating that adoptive dendritic cell transfer prolongs Factor VIII expression in the Factor VIII knockout mouse.

FIG. 10 is a chart demonstrating that five of eight mice injected with the HDAd_{Tol}-treated DC expressed levels of 10-100% normal (i.e. therapeutically relevant values) Factor VIII for 24 weeks, whereas control mice lost all detectible Factor VIII expression by week 3.

FIG. 11 is a chart demonstrating that DCₜₒₗ suppresses antibody response to repeated FVIII protein infusion.

FIG. 12 is a schematic of the experimental design for assessing the ability of the modified DCs to mediate targeted immune suppression in vivo.

FIG. 13 is a chart demonstrating that adoptive DC transfer suppressed the development of anti-albumin antibody titer.

### DETAILED DESCRIPTION

The present invention encompasses compositions and methods for inducing immunosuppression and tolerance as defined by suppression of an immune response to an antigen. In one aspect, the invention includes a genetically-modified dendritic cell (DC) that is capable of inducing tolerance in an antigen specific manner. Preferably, DC is genetically modified to express at least two immunosuppressive molecules.

The invention also provides a method of generating tolergenic DCs whereby the tolerogenic DCs are able to suppress immunity in an antigen specific fashion. In another aspect, the tolergenic DCs are able to induce T cell apoptosis and increase the frequency of antigen-specific regulatory T cells. The tolergenic DCs also provide for a method of cell therapy for antigen-targeted immune suppression to facilitate long-term therapy irrespective of method of protein delivery and/or expression. For example, the cell therapy can be used to suppress anticipated, unwanted immune responses to prolong gene therapy or recurrent infusion of therapeutic proteins.

In addition, the present invention provides a method for enhancing tolerance in a mammalian host to prolong foreign graft survival in the host and for ameliorating inflammatory-related diseases, such as autoimmune diseases, including, but not limited to, autoimmune arthritis, autoimmune diabetes, asthma, septic shock, lung fibrosis, glomerulonephritis, artherosclerosis, as well as AIDS, and the like.

The present invention includes a method of improving the presence of an exogenous protein in a mammal. In some instances, the protein is expressed in a mammalian host by way of a vector. In other instances it is applied exogenously to the mammalian host. In any event, the DC of the present invention is useful for suppressing an immune response against the exogenous protein. Therefore, the invention encompasses improving the presence of a therapeutic protein in a mammal by way of DC mediated suppression of the immune response in an antigen specific manner with respect to the therapeutic protein.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, to “alleviate” a disease means reducing the severity of one or more symptoms of the disease.

“Allogeneic” refers to a graft derived from a different animal of the same species.

“Alloantigen” is an antigen that differs from an antigen expressed by the recipient.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“An antigen presenting cell” (APC) is a cell that is capable of activating T cells, and includes, but is not limited to, monocytes/macrophages, B cells and dendritic cells (DCs).

“Antigen-loaded APC” or an “antigen-pulsed APC” includes an APC, which has been exposed to an antigen and activated by the antigen. For example, an APC may become Ag-loaded in vitro, e.g., during culture in the presence of an antigen. The APC may also be loaded in vivo by exposure to an antigen. An “antigen-loaded APC” is traditionally prepared in one of two ways: (1) small peptide fragments, known as antigenic peptides, are “pulsed” directly onto the outside of the APCs; or (2) the APC is incubated with whole proteins or protein particles which are then ingested by the APC. These proteins are digested into small peptide fragments by the APC and are eventually transported to and presented on the APC surface. In addition, the antigen-loaded APC can also be generated by introducing a polynucleotide encoding an antigen into the cell.

The term “dendritic cell” or “DC” refers to any member of a diverse population of morphologically similar cell types found in lymphoid or non-lymphoid tissues. These cells are characterized by their distinctive morphology, high levels of surface MHC-class II expression, and ability to regulate the immune response. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes.

The term “autoimmune disease” as used herein is defined as a disorder that results from an autoimmune response. An autoimmune disease is the result of an inappropriate and excessive response to a self-antigen. Examples of autoimmune diseases include, but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, and type I diabetes mellitus, among others.

As used herein, the term “autologous” is meant to refer to any material derived from the same individual to which it is later to be re-introduced into the mammal.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “DNA” as used herein is defined as deoxyribonucleic acid.

“Donor antigen” refers to an antigen expressed by the donor tissue to be transplanted into the recipient.

“Recipient antigen” refers to a target for the immune response to the donor antigen.

As used herein, an “effector cell” refers to a cell which mediates an immune response against an antigen. An example of an effector cell includes, but is not limited to a T cell and a B cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

By the term “effective amount”, as used herein, is meant an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide of the present invention can be an epitope.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “helper T cell” as used herein is defined as an effector T cell whose primary function is to promote the activation and functions of other B and T lymphocytes and or macrophages. Most helper T cells are CD4 T-cells.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immunostimulatory” is used herein to refer to increasing overall immune response.

The term “immunosuppressive” is used herein to refer to reducing overall immune response. In some instances, it is desirable to induce an antigen specific immunosuppressive effect.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “polypeptide” as used herein is defined as a chain of amino acid residues, usually having a defined sequence. As used herein the term polypeptide is mutually inclusive of the terms “peptide” and “protein”.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

As used herein, “specifically binds” refers to the fact that a first composition binds preferentially with a second composition and does not bind in a significant amount to other compounds present in the sample.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances.

“Tolerance” refers to a state characterized by the absence of a significant immune response to for example a therapeutic polypeptide. The induction of tolerance does not mean that the immune system of a subject is incapable of generating an immune response against a therapeutic polypeptide, but rather that the subject's immune system is rendered unresponsive to the presence of the therapeutic polypeptide after gene or protein delivery.

A “therapeutic polypeptide” is a polypeptide or protein that can elicit a desired therapeutic response.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “T-cell” as used herein is defined as a thymus-derived cell that participates in a variety of cell-mediated immune reactions.

The term “B-cell” as used herein is defined as a cell derived from the bone marrow and/or spleen. B cells can develop into plasma cells which produce antibodies.

As used herein, a “therapeutically effective amount” is the amount of a therapeutic composition sufficient to provide a beneficial effect to a mammal to which the composition is administered.

As used herein, to “treat” means reducing the frequency with which symptoms of a disease (i.e., viral infection, tumor growth and/or metastasis) are experienced by a patient.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “virus” as used herein is defined as a particle consisting of nucleic acid (RNA or DNA) enclosed in a protein coat, with or without an outer lipid envelope, which is capable of replicating within a whole cell.

“Xenogeneic” refers to a graft derived from an animal of a different species.

### DESCRIPTION

The present invention relates to the discovery that a DC genetically modified to express at least two immunosuppressive molecules can induce tolerance to non-harmful self antigen, a transplant, or a therapeutic protein. Thus, the present invention provides a method of enhancing the tolerogenic potential of a DC (also referred herein as tolerogenic DCs). In some instances, the DCs can be primed with an antigen to generate a tolerogenic DC capable of inducing tolerance in an antigen specific manner. In another instance, DCs can be directly induced to express the antigen of interest. For example, the antigenic specific tolerance is useful in protein therapy including, but is not limited to Factor VIII, insulin, thrombopoietin (TPO), erythropoietin (EPO), interferon-β (INF-β), INF-α, GM-CSF, tissue plasminogen activator, myelin basic protein (MBP), AXO, and antibody therapies.

The tolerogenic DCs of the present invention are useful for prolonging foreign graft survival in a mammalian host and for ameliorating inflammatory-related diseases, such as autoimmune diseases. The tolerogenic DCs are also useful for suppressing an immune response in the context of gene therapy of a desired gene or exogenous protein-based therapy. For example, the invention encompasses DC mediated suppression of the immune response against an exogenous gene to promote long-term gene expression of the gene. DC mediated suppression of the immune response can also be applied to suppression of the immune response to promote long term presence of a therapeutic protein in a mammal, for example in the context of protein therapy.

Accordingly, the present invention encompasses methods and compositions for reducing and/or eliminating an immune response to a transplant in a recipient by treating the recipient with an amount of DCs of the present invention to reduce or inhibit host rejection of the transplant. Transplant refers to any material that is to be administered to a host. For example, a transplant includes, but is not limited a biocompatible lattice, a donor tissue, an organ, a cell, a nucleic acid material, and a polypeptide.

Also encompassed are methods and compositions for reducing and/or eliminating an immune response in a host by the foreign transplant against the host, i.e., graft versus host disease, by treating the donor transplant and/or recipient of the transplant tolerogenic DC in order to inhibit or reduce an adverse response by the donor transplant against the recipient.

In addition, the present invention encompasses methods and compositions for reducing and/or eliminating an immune response to an exogenously delivered protein in a recipient by treating the recipient with an amount of the DCs of the present invention to reduce or inhibit rejection of the protein.

### Vectors and Genetically Modified Cells

The DCs of the invention can be generated by transducing the cells with a vector that results in increased expression of an immunosuppressive molecule. Any of a variety of methods well known to one of skill in the art can be used to transduce the DCs. Preferably, the DCs are transduced with a helper-dependent adenoviral vector.

The invention includes a vector comprising an isolated nucleic acid encoding an immunosuppressive molecule, wherein the immunosuppressive molecule includes, but is not limited to, a cytokine, such as, for example interleukin 2 (IL-2), interleukin 4 (IL-4), interleukin-6 (IL-6), interleukin 10 (IL-10), interferon γ, macrophage migration inhibitory factor (MIF), lymphotoxin β (LTB) and transforming growth factor β (TGFβ). The nucleic acid encoding an immunosuppressive molecule is operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The nucleic acid encoding an immunosuppressive molecule of the invention can be cloned into a number of types of vectors. However, the present invention should not be construed to be limited to any particular vector. Instead, the present invention should be construed to encompass a wide plethora of vectors which are readily available and well-known in the art. For example, an isolated nucleic acid encoding an immunosuppressive molecule of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector, and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. Preferably, the virus is helper-dependent adenovirus (HD-Ad). In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the immunosuppressive molecule, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the immunosuppressive molecule, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction, sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a cytokine signaling regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Generation of an Antigen Specific (Pulsed) Tolerogenic DC

The invention includes a genetically modified DC expressing at least two immunosuppressive molecules that can further be exposed or otherwise “pulsed” or “primed” with an antigen. For example, the tolerogenic DC may become “antigen-loaded” in vitro, e.g., by culture ex vivo in the presence of an antigen, or directly genetically modified to express a desirable antigen, or in vivo by exposure to an antigen.

A skilled artisan would also readily understand that the tolerogenic DC can be “pulsed” in a manner that exposes the DC to an antigen for a time sufficient to promote presentation of that antigen on the surface of the DC. For example, DCs can be exposed to an antigen where the antigen is in a form of a small peptide fragment, known as antigenic peptide. The antigenic peptide is “pulsed” directly onto the outside of the DC; or the DCs can be incubated with whole proteins or protein particles which are then ingested by the DCs. These whole proteins are digested into small peptide fragments by the DC and eventually carried to and presented on the DC surface. Antigen in peptide form may be exposed to the cell by standard “pulsing” techniques described herein. The antigen may also be mixed in nature being derived from tissue and cell extracts.

Without wishing to be bound by any particular theory, the antigen in the form of a foreign or an autoantigen is processed by the DC of the invention in order to retain the immunogenic form of the antigen. The immunogenic form of the antigen implies processing of the antigen through fragmentation to produce a form of the antigen that can be recognized by and stimulate immune cells, for example T cells. Preferably, such a foreign or an autoantigen is a protein which is processed into a peptide by the DC. The relevant peptide which is produced by the DC may be extracted and purified for use as an immunogenic composition. Peptides processed by the DC may also be used to induce tolerance to the proteins processed by the DC.

It is believed that autoimmune diseases result from an immune response being directed against “self-proteins,” otherwise known as autoantigens, i.e., autoantigens that are present or endogenous in a mammal. In an autoimmune response, these “self-proteins” are presented to T cells which cause the T cells to become “self-reactive.” According to the method of the invention, DC are pulsed with an antigen to produce the relevant “self-peptide.” The relevant self-peptide is different for each individual because MHC products are highly polymorphic and each individual MHC molecule might bind different peptide fragments. The “self-peptide” can then be used to design competing peptides or to induce tolerance to the self protein in the mammal in need of treatment. In the context of protein-based therapy, the DC can be primed with the protein or an antigenic portion thereof.

The antigen-activated DC, otherwise known as a “pulsed DC”, is produced by exposure of the DC to an antigen either in vitro or in vivo. In the case where the DC is pulsed in vitro, the DC is plated on a culture dish and exposed to an antigen in a sufficient amount and for a sufficient period of time to allow the antigen to bind to the DC. The amount and time necessary to achieve binding of the antigen to the DC may be determined by using methods known in the art or otherwise disclosed herein. Other methods known to those of skill in the art, for example immunoassays or binding assays, may be used to detect the presence of antigen on the DC following exposure to the antigen.

In a further embodiment of the invention, the DC may be genetically modified using a vector which allows for the expression of a specific protein by the DC. The protein which is expressed by the DC may then be processed and presented on the cell surface on an MHC receptor. The modified DC may then be used as an immunogenic composition to induce tolerance to the protein.

As discussed elsewhere herein, vectors may be prepared to include a specific polynucleotide which encodes and expresses a desired protein. Preferably, retroviral or lentiviral vectors are used to infect the cells. More preferably, adenoviral vectors are used to infect the cells.

As discussed elsewhere herein, various methods can be used for transfecting a polynucleotide into a host cell. The methods include, but are not limited to, calcium phosphate precipitation, lipofection, particle bombardment, microinjection, electroporation, colloidal dispersion systems (i.e. macromolecule complexes, nanocapsules, microspheres, beads, and lipid-based systems including oil-in-water emulsions, micelles, mixed micelles, and liposomes).

Various types of vectors and methods of introducing nucleic acids into a cell are discussed elsewhere herein. For example, a vector encoding an antigen may be introduced into a host cell by any method in the art. For example, the expression vector can be transferred into a host cell by physical, chemical or biological means. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). It is readily understood that the introduction of the expression vector comprising a polynucleotide encoding an antigen yields a pulsed cell.

The antigen may be derived from a virus, a fungus, or a bacterium. The antigen may be a self-antigen or an antigen associated with a disease selected from the group consisting of an infectious disease, a cancer, genetic disease, an autoimmune disease. The antigen may be a therapeutic protein exogenously produced to achieve a pharmacological or biological effect in the recipient.

The invention includes a cellular composition comprising a DC that has been modified to enhance its tolerogenic potential. The tolerogenic DC can then be further transfected with a nucleic acid encoding an antigen to generate an antigen specific tolerogenic DC. In another aspect, the DC can be pulsed with an immunostimulatory protein comprising an antigen to generate an antigen-loaded cell.

### Therapeutic Application

The invention includes a method of suppressing an immune response in a mammal for the treatment or prevention of an autoimmune condition or transplantation rejection to include organ, cell, and/or protein transplantation. As discussed in more detail below, the DCs of the invention are useful in gene and protein therapy. In any event, the present invention includes a method of using genetically modified DCs to express at least two immunosuppressive molecules (tolerogenic DCs) as a therapy to modulate the immune response. In some instances, the DCs are further modified (e.g., primed to a specific antigen) to generate a DC having tolerogenic potential in an antigen specific manner. The invention is based on the discovery that tolerogenic DC can induce tolerance.

The present invention includes a method of using genetically modified DCs to express at least two immunosuppressive molecules (tolerogenic DCs) as a therapy to modulate the immune response. In some instances, the tolerogenic DCs are able to induce T cell apoptosis and increase the frequency of antigen-specific regulatory T cells.

In some instances, the invention is useful in avoiding or suppressing side effects resulting from the patient's immune response mounted against the drug and/or protein administered to the patient, which therefore decreases the efficacy and safety of the drug and/or protein. The DCs of the invention are useful to suppress the immune response against the therapeutic protein.

In a non-limiting example, the tolerogenic DCs can be used in an adoptive transfer strategy to suppress the immune response to FVIII gene therapy. Without wishing to be bound by any particular theory, adoptive transfer of FVIII-loaded, tolerogenic DC are used to induce suppression of the anti-FVIII immune response and thereby prolong transgene expression of FVIII. However, the invention should not be limited to using tolergenic DC with FVIII gene therapy. Rather, the tolerogenic DCs can be used for any desired transgene in the context of gene therapy or protein therapy. This is because the tolerogenic DC can be exposed to any desirable transgene or otherwise rendered antigen specific to the transgene and therefore can suppress an immune response to the corresponding transgene in an antigen specific manner. For example, the antigenic specific tolerance is useful in protein therapy or otherwise known as protein therapeutics. In some instances, the invention is applicable to any type of therapy where the therapy is known to elicit an antibody response (e.g., protein therapy with anti-protein antibody response).

Protein therapeutics include, but is not limited to monoclonal antibodies, enzymes, cytokines, and toxins. An example of monoclonal antibodies that is desirable to target using the present invention is humanized antibodies, such as Remicade. The invention is also applicable to therapy using fusion proteins with artificial activities. For example, Enbrel is a fusion of the extracellular domain of a TNF receptor with an IgG1 Fc region. Enbrel is used to treat rheumatoid arthritis, and is believed to function by titrating TNF and preventing TNF action. However, a significant incidence of anti-Enbrel antibodies have been noted in patients treated with Enbrel.

Another example of a therapeutically useful class of fusion proteins is the immunocytokines. These proteins include an antibody moiety and a cytokine moiety, and are useful for targeting cytokines to diseased cells, such as cancer cells. However, the therapeutic use of many of these fusion proteins is reduced due to their immunogenicity in mammals, especially humans.

The present invention is applicable to immunogenicity of protein therapeutics including but is not limited to human thrombopoietin (TPO), erythropoietin (EPO), interferon-β (INF-β), INF-α, GM-CSF, human tissue plasminogen activator, myelin basic protein (MBP), AXO, and the likes. The invention is also applicable to enhancing the therapeutic effect of enzyme replacement therapy including, but not limited to Cerezyme, Fabrizyme, and the like.

Accordingly, the invention relates to general cell therapy for antigen-targeted immune suppression to facilitate long-term therapy. The present invention includes a method of suppressing anticipated, unwanted immune responses to prolong gene/protein therapy. The tolerogenic DC can be administered to the recipient, prior to, at the same time, or a short time after undergoing gene/protein therapy.

The present invention encompasses a method of reducing and/or eliminating an immune response to a transplant in a recipient by administering to the recipient of the transplant an amount of tolerogenic DCs effective to reduce or inhibit host rejection of the transplant. Without wishing to be bound to any particular theory, the DCs that are administered to the recipient of the transplant inhibit the activation and proliferation of the recipient's T cells or induce tolerance.

The transplant can include a biocompatible lattice or a donor tissue, organ, cell or molecule, to be transplanted. An example of a transplant may include but is not limited to skin cells or tissue, bone marrow, and solid organs such as heart, pancreas, kidney, lung and liver. In some instances, the transplant is a nucleic acid or a protein.

Based upon the disclosure provided herein, DCs can be obtained from any source, for example, from the tissue donor, the transplant recipient or an otherwise unrelated source (a different individual or species altogether). The DCs may be autologous with respect to the T cells (obtained from the same host) or allogeneic with respect to the T cells. In the case where the DCs are allogeneic, the DCs may be autologous with respect to the transplant to which the T cells are responding to, or the DCs may be obtained from a mammal that is allogeneic with respect to both the source of the T cells and the source of the transplant to which the T cells are responding to. In addition, the DCs may be xenogeneic to the T cells (obtained from an animal of a different species), for example rat DCs may be used to suppress activation and proliferation of human T cells.

Another embodiment of the present invention encompasses the route of administering DCs to the recipient of the transplant. DCs can be administered by a route which is suitable for the placement of the transplant, i.e. a biocompatible lattice or a donor tissue, organ or cell, nucleic acid or protein, to be transplanted. DCs can be administered systemically, i.e., parenterally, by intravenous injection or can be targeted to a particular tissue or organ, such as bone marrow. DCs can be administered via a subcutaneous implantation of cells or by injection of the cells into connective tissue, for example, muscle.

DCs can be suspended in an appropriate diluent, at a concentration of from about 0.01 to about 5×10⁶ cells/ml. Suitable excipients for injection solutions are those that are biologically and physiologically compatible with the DCs and with the recipient, such as buffered saline solution or other suitable excipients. The composition for administration can be formulated, produced and stored according to standard methods complying with proper sterility and stability.

The dosage of the DCs varies within wide limits and may be adjusted to the mammal requirements in each particular case. The number of cells used depends on the weight and condition of the recipient, the number and/or frequency of administrations, and other variables known to those of skill in the art.

Between about 10⁵ and about 10¹³ DCs per 100 kg body weight can be administered to the mammal. In some embodiments, between about 1.5×10⁶ and about 1.5×10¹² cells are administered per 100 kg body weight. In some embodiments, between about 1×10⁹ and about 5×10¹¹ cells are administered per 100 kg body weight. In some embodiments, between about 4×10⁹ and about 2×10¹¹ cells are administered per 100 kg body weight. In some embodiments, between about 5×10⁸ cells and about 1×10¹⁰ cells are administered per 100 kg body weight.

In another embodiment of the present invention, DCs are administered to the recipient prior to, or contemporaneously with a transplant to reduce and/or eliminate host rejection of the transplant. While not wishing to be bound to any particular theory, DCs can be used to condition a recipient's immune system to the transplant by administering DCs to the recipient, prior to, or at the same time as transplantation of the transplant, in an amount effective to reduce, inhibit or eliminate an immune response against the transplant by the recipient's T cells. The DCs affect the T cells of the recipient such that the T cell response is reduced, inhibited or eliminated when presented with the transplant. Thus, host rejection of the transplant may be avoided, or the severity thereof reduced, by administering DCs to the recipient, prior to, or at the same time as transplantation.

In yet another embodiment, DCs can be administered to the recipient of the transplant after the administration of the transplant. Further, the present invention comprises a method of treating a patient who is undergoing an adverse immune response to a transplant by administering DCs to the patient in an amount effective to reduce, inhibit or eliminate the immune response to the transplant, also known as host rejection of the transplant.

### Therapy to Inhibit Graft Versus Host Disease Following Transplantation

The present invention includes a method of using DCs as a therapy to inhibit graft versus host disease following transplantation. Accordingly, the present invention encompasses a method of contacting a donor transplant, for example a biocompatible lattice or a donor tissue, organ or cell, with DCs prior to transplantation of the transplant into a recipient. The DCs serve to ameliorate, inhibit or reduce an adverse response by the donor transplant against the recipient.

As discussed elsewhere herein, DCs can be obtained from any source, for example, from the tissue donor, the transplant recipient or an otherwise unrelated source (a different individual or species altogether) for the use of eliminating or reducing an unwanted immune response by a transplant against a recipient of the transplant. Accordingly, DCs can be autologous, allogeneic or xenogeneic to the tissue donor, the transplant recipient or an otherwise unrelated source.

In an embodiment of the present invention, the transplant is exposed to DCs prior to transplantation of the transplant into the recipient. In this situation, an immune response against the transplant caused by any alloreactive recipient cells would be suppressed by the DCs present in the transplant. The DCs are allogeneic to the recipient and may be derived from the donor or from a source other than the donor or recipient. In some cases, DCs autologous to the recipient may be used to suppress an immune response against the transplant. In another case, the DCs may be xenogeneic to the recipient, for example mouse or rat DCs can be used to suppress an immune response in a human. However, it is preferable to use human DCs in the present invention.

In another embodiment of the present invention, the donor transplant can be “preconditioned” or “pretreated” by treating the transplant prior to transplantation into the recipient in order to reduce the immunogenicity of the transplant against the recipient, thereby reducing and/or preventing graft versus host disease. The transplant can be contacted with cells or a tissue from the recipient prior to transplantation in order to activate T cells that may be associated with the transplant. Following the treatment of the transplant with cells or a tissue from the recipient, the cells or tissue may be removed from the transplant. The treated transplant is then further contacted with DCs in order to reduce, inhibit or eliminate the activity of the T cells that were activated by the treatment of the cells or tissue from the recipient. Following this treatment of the transplant with DCs, the DCs may be removed from the transplant prior to transplantation into the recipient. However, some DCs may adhere to the transplant, and therefore, may be introduced to the recipient with the transplant. In this situation, the DCs introduced into the recipient can suppress an immune response against the recipient caused by any cell associated with the transplant. Without wishing to be bound to any particular theory, the treatment of the transplant with DCs prior to transplantation of the transplant into the recipient serves to reduce, inhibit or eliminate the activity of the activated T cells, thereby preventing restimulation, or inducing hyporesponsiveness of the T cells to subsequent antigenic stimulation from a tissue and/or cells from the recipient. One skilled in the art would understand based upon the present disclosure, that preconditioning or pretreatment of the transplant prior to transplantation may reduce or eliminate the graft versus host response.

For example, in the context of bone marrow or peripheral blood stem cell (hematopoietic stem cell) transplantation, attack of the host by the graft can be reduced, inhibited or eliminated by preconditioning the donor marrow by using the pretreatment methods disclosed herein in order to reduce the immunogenicity of the graft against the recipient. As described elsewhere herein, a donor marrow can be pretreated with DCs from any source, preferably with recipient DCs in vitro prior to the transplantation of the donor marrow into the recipient. In a preferred embodiment, the donor marrow is first exposed to recipient tissue or cells and then treated with DCs. Although not wishing to be bound to any particular theory, it is believed that the initial contact of the donor marrow with recipient tissue or cells function to activate the T cells in the donor marrow. Treatment of the donor marrow with the DCs induces hyporesponsiveness or prevents restimulation of T cells to subsequent antigenic stimulation, thereby reducing, inhibiting or eliminating an adverse affect induced by the donor marrow on the recipient.

In an embodiment of the present invention, a transplant recipient suffering from graft versus host disease may be treated by administering DCs to the recipient to reduce, inhibit or eliminate the severity thereof from the graft versus host disease where the DCs are administered in an amount effective to reduce or eliminate graft versus host disease.

In this embodiment of the invention, preferably, the recipient's DCs may be obtained from the recipient prior to the transplantation and may be stored and/or expanded in culture to provide a reserve of DCs in sufficient amounts for treating an ongoing graft versus host reaction. However, as discussed elsewhere herein, DCs can be obtained from any source, for example, from the tissue donor, the transplant recipient or an otherwise unrelated source (a different individual or species altogether).

### Advantages of Using DCs

Based upon the disclosure herein, it is envisioned that the DCs of the present invention can be used in conjunction with current modes, for example the use of immunosuppressive drug therapy, for the treatment of host rejection to the donor tissue or graft versus host disease. An advantage of using DCs in conjunction with immunosuppressive drugs in transplantation is that by using the methods of the present invention to ameliorate the severity of the immune response in a transplant recipient, the amount of immunosuppressive drug therapy used and/or the frequency of administration of immunosuppressive drug therapy can be reduced. A benefit of reducing the use of immunosuppressive drug therapy is the alleviation of general immune suppression and unwanted side effects associated with immunosuppressive drug therapy.

It is also contemplated that the cells of the present invention may be administered into a recipient as a “one-time” therapy for the treatment of host rejection of donor tissue or graft versus host disease. A one-time administration of DCs into the recipient of the transplant eliminates the need for chronic immunosuppressive drug therapy. However, if desired, multiple administrations of DCs may also be employed.

The invention described herein also encompasses a method of preventing or treating transplant rejection and/or graft versus host disease by administering DCs in a prophylactic or therapeutically effective amount for the prevention, treatment or amelioration of host rejection of the transplant and/or graft versus host disease. Based upon the present disclosure, a therapeutic effective amount of DCs is an amount that inhibits or decreases the number of activated T cells, when compared with the number of activated T cells in the absence of the administration of DCs. In the situation of host rejection of the transplant, an effective amount of DCs is an amount that inhibits or decreases the number of activated T cells in the recipient of the transplant when compared with the number of activated T cells in the recipient prior to administration of the DCs. In the case of graft versus host disease, an effective amount of DCs is an amount that inhibits or decreases the number of activated T cells present in the transplant.

An effective amount of DCs can be determined by comparing the number of activated T cells in a recipient or in a transplant prior to the administration of DCs thereto, with the number of activated T cells present in the recipient or transplant following the administration of DCs thereto. A decrease, or the absence of an increase, in the number of activated T cells in the recipient of the transplant or in the transplant itself that is associated with the administration of DCs thereto, indicates that the number of DCs administered is a therapeutic effective amount of DCs.

The invention also includes methods of using DCs of the present invention in conjunction with current mode, for example the use of immunosuppressive drug therapy, for the treatment of host rejection to the donor tissue or graft versus host disease. An advantage of using tolerogenic DCs in conjunction with immunosuppressive drugs in transplantation is that by using the methods of the present invention to ameliorate the severity of the immune response following transplantation, the amount of immunosuppressive drug therapy used and/or the frequency of administration of immunosuppressive drug therapy can be reduced. A benefit of reducing the use of immunosuppressive drug therapy is the alleviation of general immune suppression and unwanted side effects associated with immunosuppressive drug therapy.

### Gene Therapy

Gene therapy can be used to replace genes that are defective in a mammal. The invention may also be used to express a desired protein in a mammal. A cell can be introduced with a gene for a desired protein and introduced into a mammal within whom the desired protein would be produced and exert or otherwise yield a therapeutic effect. This aspect of the invention relates to gene therapy in which therapeutic proteins are administered to a mammal by way of introducing a genetically modified cell into a mammal. The genetically modified cells are implanted into a mammal who will benefit when the protein is expressed by the cells in the mammal. In some instances, the genetically modified DCs are implanted into a mammal who will benefit when the protein is expressed and secreted by the cells in the mammal.

According to the present invention, gene constructs which comprise nucleotide sequences that encode heterologous proteins are introduced into a cell. That is, the cells are genetically altered to introduce a gene whose expression has therapeutic effect in the mammal. According to some aspects of the invention, cells from a mammal or from another mammal or from a non-human animal may be genetically altered to replace a defective gene and/or to introduce a gene whose expression has therapeutic effect in the mammal.

In all cases in which a gene construct is transfected into a cell, the heterologous gene is operably linked to regulatory sequences required to achieve expression of the gene in the cell. Such regulatory sequences include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

In some aspects of the invention, a mammal suffering from a disease, disorder, or a condition that is characterized by a genetic defect or a defect associated with decreased level of expression of a particular gene may be treated by supplementing, augmenting and/or replacing defective or deficient cells with cells that correctly express a normal gene.

Where the lack or decreased level of expression of a particular protein causes a disease or condition associated with such expression, an equivalent recombinant protein can be administered to the mammal in need thereof. The recombinant protein can be directly administered to the mammal. Alternatively, the recombinant protein can be expressed from a construct comprising a nucleic acid encoding the protein. In any event, the present invention provides an improvement to gene therapy. This is because there are situations where unwanted immune responses occur against a therapeutic protein or to the nucleic acid construct encoding the protein. The present invention provides a method of enhancing the expression of the therapeutic protein by way of inhibiting or suppressing an immune response against the therapeutic protein or the nucleic acid construct encoding the protein.

An exogenous protein foreign to the recipients such as an antibody or other such protein maybe given to affect a specific disease process not related per se with the deficiency of the therapeutic protein. In this scenario, the recipient immune response can also reject this treatment. Treatment with tolerogenic DCs specific for the protein therapy will decrease toxicity of the treatment and prolong efficacy of treatment by preventing immunological rejection of said treatment.

The tolerogenic DCs can be further modified to a particular antigen, wherein the antigen primes the DC to the desired therapeutic protein or to the nucleic acid construct encoding the protein. The primed tolerogenic DC is useful to specifically suppress or induce tolerance against the therapeutic protein or to the nucleic acid construct encoding the protein. The DCs of the present invention allows for long-term gene expression of an exogenous gene due to suppression of an immune response against a specific gene and/or protein product of that gene.

### Protein Therapy

In addition to the gene therapy aspect of the invention, the tolerogenic DCs are equally useful in the context of protein-based therapy. The desired protein or therapeutic protein can be made by any means in the art. For example, a host cell transfected with a nucleic acid vector directing expression of a nucleotide sequence encoding a desired protein can be cultured in a medium under appropriate conditions to allow expression of the protein to occur. Protein can be isolated from cell culture medium, host cells, or both using techniques known in the art for purifying proteins. Once purified, partially or to homogeneity, the recombinantly produced protein or portions thereof can be utilized in compositions suitable for pharmaceutical administration as described in detail herein. The therapeutic protein can also be a synthetically derived peptide or polypeptide with the purpose of directing the immune response toward the antigen and tolerogenic effect.

The basic approach of using DCs of the invention to induce tolerance has widespread application as an adjunct to therapies which utilize a potentially immunogenic molecule for therapeutic purposes. For example, an increasing number of therapeutic approaches utilize a proteinaceous molecule, such as an antibody, fusion protein or the like, for treatment of a clinical disorder. A limitation to the use of such molecules therapeutically is that they can elicit an immune response directed against the therapeutic molecule in the subject being treated (e.g., the efficacy of Factor VIII in human subjects is hindered by the induction of an immune response against Factor VIII in the human subject). The present invention is an improvement on conventional protein therapy in the context of inducing tolerance against the administered molecule. Preferably, the tolerogenic effect is specific to antigen or otherwise specific to the administered molecule thereby enhancing the period of time that the molecule is present in the recipient.

By way of example, Factor VIII is discussed as a representative type of protein therapy. However, any candidate protein can be applied to the present invention. Hemophilia A is caused by deficiencies in the expression or function of clotting factor VIII (FVIII). Treatment of hemophilia currently involves infusion of normal FVIII protein obtained from plasma concentrates or as purified from cultured cells engineered to express recombinant FVIII protein. Therapeutic benefit is achieved by restoration of plasma levels to 5-10% of normal plasma levels (200-300 ng or 1 unit per milliliter). Studies have shown that maintenance of greater than 10-30% of the normal plasma levels allows for a near normal lifestyle. The use of the tolerogenic DCs of the present invention provides a method of increasing the success of FVIII protein therapy by way of decreasing an unwanted immune response against FVIII.

Given the role of the tolerogenic DCs of the invention, this aspect of the invention provides a method for inducing tolerance against the therapeutic protein. The tolerogenic potential of the DCs can result in more effective downregulation of immune responses in vivo without unwanted side effects (e.g., complement activation, antibody-dependent cellular cytotoxicity, etc.). Downregulation of an immune response by DCs of the invention may be in the form of inhibiting or blocking an immune response already in progress or may involve preventing the induction of an immune response. The functions of activated T cells, such as T cell proliferation and cytokine (e.g., IL-2) secretion, may be inhibited by suppressing T cell responses or by inducing specific tolerance in T cells, or both. Immunosuppression of T cell responses is generally an active process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, T cell unresponsiveness or anergy can be demonstrated by the lack of a T cell response upon reexposure to specific antigen in the absence of the tolerizing agent.

Administration of a tolerogenic DC of the invention to inhibit antigen-specific T cell responses can be applied to these therapeutic situations to enable long term usage of the therapeutic molecule in the subject without elicitation of an immune response. For example, a therapeutic protein (e.g., Factor VIII) is administered to a subject (e.g., human), which typically activates an immune response for Factor VIII in the subject. To inhibit the immune response against Factor VIII, Factor VIII is administered to the subject together with an effect amount of tolerogenic DC of the invention. Preferably, the tolerogenic DC has been primed with Factor VIII or an antigenic portion thereof in order to generate tolerance specifically directed to Factor VIII (e.g., an antigen specific tolerance). The invention should not be limited to only Factor VIII therapy, but rather the invention should include all types of protein therapy, for example insulin therapy or antibody therapies.

As discussed elsewhere herein, inhibition of T cell responses by a tolerogenic DCs of the invention is useful in situations of cellular, tissue, skin and organ transplantation and in bone marrow transplantation (e.g., to inhibit graft-versus-host disease) as well as gene and protein therapy. In the context of protein therapy, induction of tolerance can result in reduced protein destruction for example by way of an unwanted antibody response against the administered protein. Induction of antigen-specific tolerance can result in long-term existence of the therapeutic protein without the need for generalized immunosuppression.

It should be understood that the methods described herein may be carried out in a number of ways and with various modifications and permutations thereof that are well known in the art. It may also be appreciated that any theories set forth as to modes of action or interactions between cell types should not be construed as limiting this invention in any manner, but are presented such that the methods of the invention can be more fully understood.

The following examples further illustrate aspects of the present invention. However, they are in no way a limitation of the teachings or disclosure of the present invention as set forth herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXAMPLES

The invention is now described with reference to the following Examples. These Examples are provided for the purpose of illustration only, and the invention is not limited to these Examples, but rather encompasses all variations which are evident as a result of the teachings provided herein.

Inhibitory antibodies such as antibodies directed against recombinant proteins constitute a significant clinical obstacle to protein and to gene therapies. The experiments presented herein demonstrate that transducing DC with helper-dependent adenovirus to express the immune suppressive cytokines TGF-β and IL-10 renders them tolerogenic by attenuating DC activation, inducing T cell apoptosis, and increasing the frequency of antigen-specific regulatory T cells. The transduced DC can be used in an adoptive transfer to suppress anticipated, unwanted immune responses to prolong gene therapy strategy. For example, it was observed that adoptive transfer of FVIII-loaded, tolerogenic DC to FVIII knock-out mice prior to gene transfer induced suppression of the anti-FVIII immune response, and prolonged transgene expression.

The experiments disclosed herein were conducted to explore the ability of genetically modified DCs expressing at least two immunosuppressive molecules to induce tolerance. The results disclosed herein demonstrate that long-term gene expression can be accomplished by suppressing an immune response against the exogenous gene or gene product. These findings are applicable to other protein and gene therapies, and autoimmune diseases and solid organ transplantation.

The materials and methods employed in the experiments disclosed herein are now described.

### Mice, Primary Cells and Cell Lines

C57/B6J, C3H/HeJ, BALB/cJ, C.Cg-Tg (DO11.10)10Dlo/J, and FVIII knockout mice were purchased from Jackson Laboratories. All animals and protocols were used in accordance with the Baylor College of Medicine institutional animal care and use committee.

Bone marrow derived DCs (BMDC) were harvested from tibias and femurs and maintained in RPMI 1640 media plus 10% FBS, 5 mM L-glutamine, 50 μM and 2-ME, supplemented with penicillin and streptomycin as previously described (Inaba et al., 1992 J Exp Med. 176: 1693-1702). Splenocytes were collected from age and sex-matched donors of the indicated strains, and where indicated, T cells were purified by negative selection using the MACs pan T cell isolation kit (Miltenyi Biotec, Auburn, Calif.) according to manufacturer's protocol. In general, mixed-lymphocyte co-culture experiments were set up as indicated in 96-well round bottom plates, with 10⁵ DC as antigen presenting cells, with 10⁶ responder splenocytes per well. For mixed-DO11.10 T cell containing co-cultures, 2×10⁵ purified T cells were added to 8×10⁵ wild type BALB/cJ splenocytes to comprise the 10⁶ splenocyte components. In some experiments, DC antigen specificity was controlled by adding 5 ug/ml OVA (Sigma-Aldrich, St. Louis, Mo.), or 5 ug/ml human AAT (RDI, Concord Mass.). Cells were cultured for 24 hours prior to Annexin V staining and 3 days prior to surface marker flow cytometric analysis.

### Flow Cytometry

Where indicated, cells were stained with annexin V-APC and the vital dye 7-AAD, and analyzed using a FACsArray bioanalyzer according to manufacturer's instructions (BD Biosciences, San Jose, Calif.). In general co-cultures were stained with the indicated combinations of antibodies against mouse anti-CD4 (L3T4), anti-CD25 (PC61.5), and anti-FoxP3 (FJK-16s) (eBiosciences, San Diego, Calif.), in combination with DO11.10 TCRtg specific antibody (mouse anti-mouse DO11.10 TCR clone KJ1-26, Caltag laboratories, Burlingame, Calif.). In all experiments, at least 30,000 cells were analyzed in the live lymphocyte gate. When necessary, multi-color flow cytometry was performed on an LSR II analyzer (BD Biosciences) in the cytometry and cell sorting facility at BCM.

### Viral Vectors and Transduction

HD-AdGFP, and HD-Adzero were generated as previously described (Palmer et al., 2003 Mol Ther. 8: 846-852). HD-AdTGF-β/IL-10 cloning and amplification is as follows. Briefly, a transgene cassette containing back-to back TGF-β and IL-10 expression cassettes driven by CMV promoters was cloned into the helper-dependent backbone, pΔ28e4 via the AscI/BssHII restriction sites. The HD-AdTGF-β/IL-10 vector plasmid was digested with PmeI to release the linear vector genome, and transfected into the packaging 293 cell line. Next, the cells were co-infected with a helper virus to trans-complement the adenoviral E1 early gene provided by the packaging cell and to assemble the helper-dependent vector. Because the packaging cells also express the site specific Cre recombinase the loxP-flanked adenoviral packaging signal is excised from the helper virus, while the transfected-HDV genome packaging signal is retained, and preferentially packaged into the nascent virions during amplification.

DC were genetically modified with HD-Ad:CaPi precipitates. Briefly, CaPi precipitates were formed by placing the indicated amount of vector into a total of 500 μl of Eagles Minimal Essential Media (EMEM) (pH 7.4) (Sigma-Aldrich) into a sterile 12×75 mm polystyrene round bottom flow cytometry tube (BD Falcon). Next, a second tube was prepared with 498 μl EMEM supplemented with 2 μl of 2 M CaCl₂ (ProFection®, Calcium-Phosphate mammalian transfection System; Promega, Madison Wis., USA). After light vortex, the contents of the calcium-containing tube was added to the vector-containing tube, light vortex, and incubated at room temperature for 30 minutes. 250 μl of the above complex was added to each well of a 24 well dish for 1 hour followed by removal by aspiration and addition of fresh media.

Adenovirus transduction-permissive 293 cells were cultured in DMEM supplemented with 10% FBS, 5 mM L-glutamine, and supplemented with penicillin and streptomycin. The adenovirus neutralization assay was similar to a previously described assay (Sprangers et al., 2003 J Clin Microbiol. 41: 5046-5052), which was performed by incubating serial dilutions of mouse serum with 50 vp/cell of a first-generation adenovirus expressing the beta-galactosidase transgene.

FVIII knockout mice were injected via the tail-vein with 5×10¹² vp/kg of a helper-dependent vector expressing the B-domain deleted human FVIII under control of the PGK promoter. Adoptive cell transfer was administered where indicated by i.p. injection of 1×10⁶ DC per recipient mouse.

### FVIII Expression, Immunization, and Antibody Titers

Plasma was collected from each FVIII KO mouse at the indicated time points and measured for the activity of FVIII by COATEST as previously described (McCormack et al., 2006 J Thromb Haemost. 2006 4: 1218-1225).

Animals immunized with human albumin (Calbiochem) were injected with 50 μg of the protein mixed with Imject® Freund's Complete Adjuvant (Pierce Biotechnology, Rockford, Ill.) according to manufacturers instructions.

Titers for anti-FVIII total IgG, and anti-albumin total IgG were measured in plasma samples collected at the indicated times by ELISA. Titers were assigned based on limiting dilution as described prebiously (Pastore et al., 1999 Hum Gene Ther. 10: 1773-1781.

### Cytokine Analysis

Cytokines measured using cytometric bead array kits for IL-6 and TNF-α were assayed on a FACSarray (BD Biosciences) bio analyzer system according to the manufacturers instructions. Quantitative analysis by traditional ELISA was performed for human TGF-β1 and IL-10 according to manufacturer's instructions (R&D Systems, Minneapolis, Minn., USA).

### Statistical Analysis

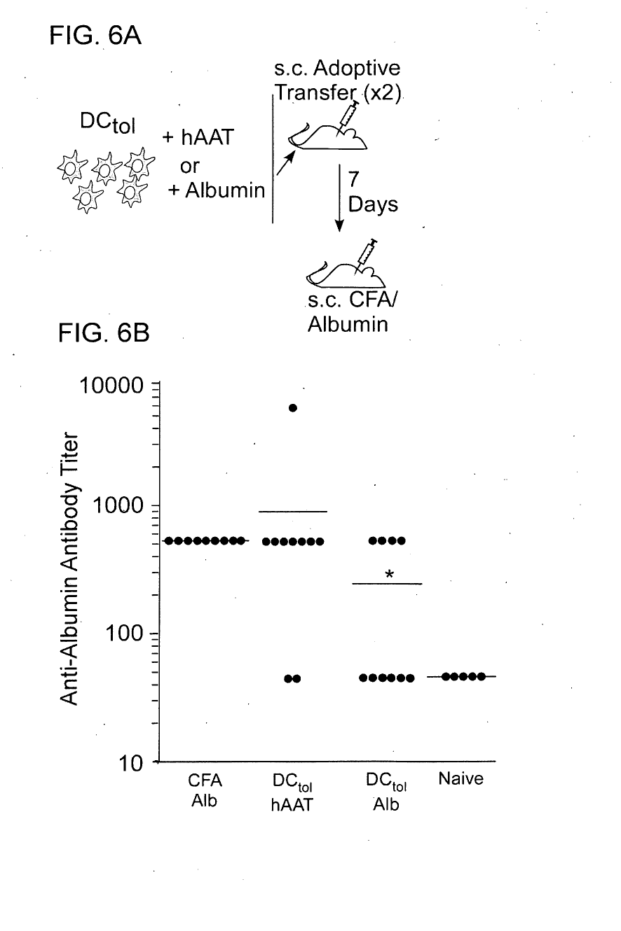

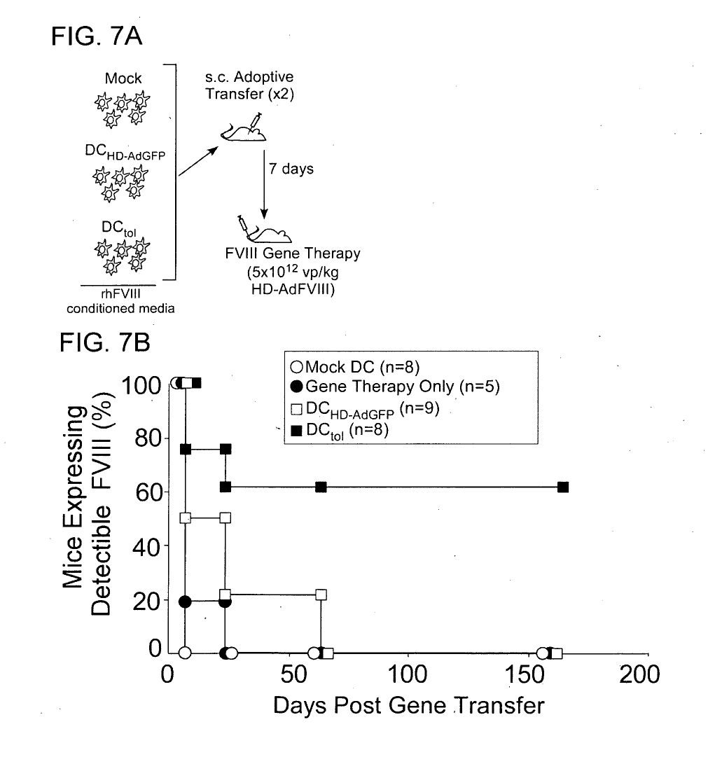

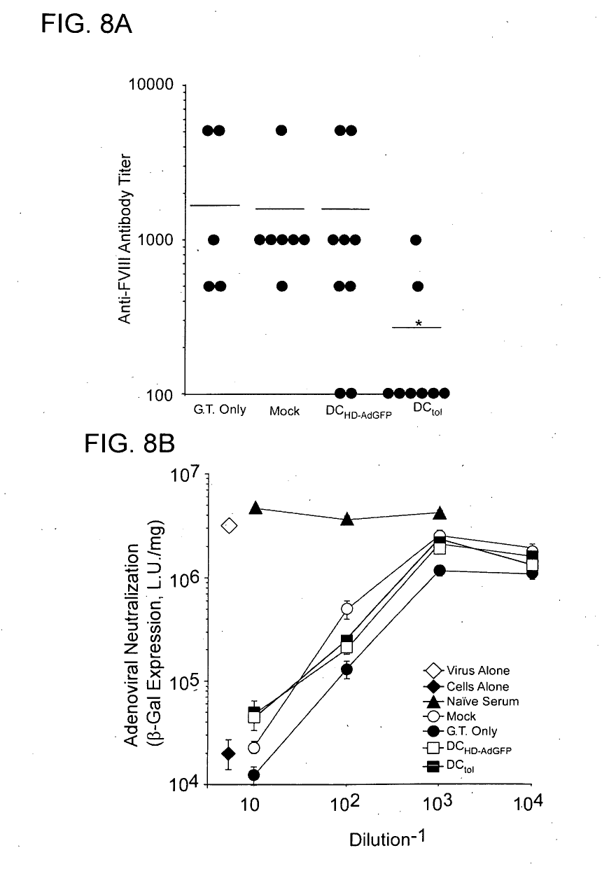

Statistical analysis in each independent in vitro experiment was performed with unpaired, two-tailed Student's t-test. FIGS. 6B and 8B show data from assigned antibody titers by limiting dilution, and statistical relevance was determined by non-parametric Freeman-Halton extension of Fisher's exact probability test for small sample sizes. In FIG. 6B, naïve treated mice were omitted from the non parametric analysis as they were not subject to immunization. The probability of mice expressing FVIII at each indicated time point in FIG. 7B was determined by non-parametric chi-square analysis. In all cases a confidence interval of 0.95 was used, and p<0.05 was considered significant.

### Example 1

### Dendritic Cell Mediated Adoptive Immune-Modulation Suppresses the FVIII Antibody Response Resulting in Long-Term Gene Expression

Genetic modification of dendritic cells (DC) is a powerful tool to harness the resulting immune response to antigens of interest. A general goal of this approach has been to induce immunity to harmful viral infections, bacteria, or tumor antigens. The results presented herein demonstrate that DCs are useful in inducing tolerance to non-harmful self antigen, transplant, or therapeutic antigens. The tolerogenic potential of DCs offers a significant improvement to current therapies.

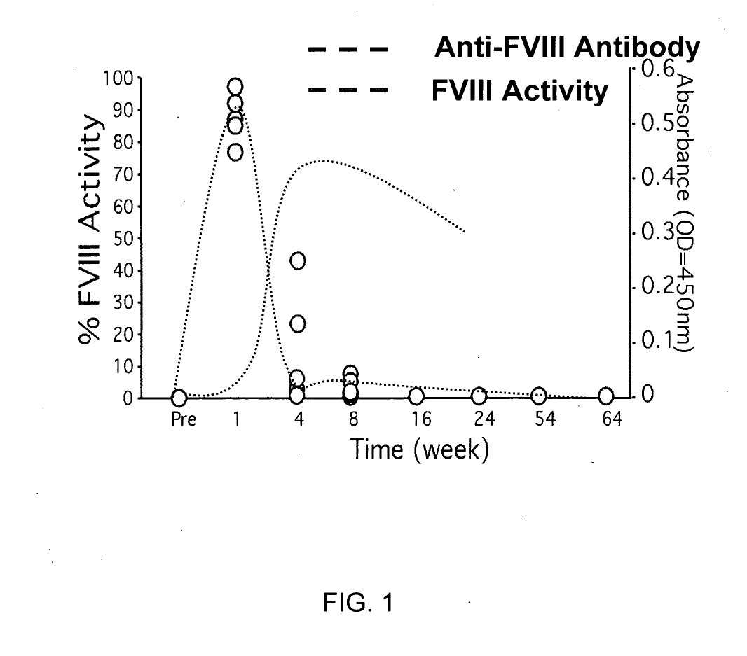

It has been demonstrated that Factor VIII gene transfer by systemic injection of helper-dependent vector resulted in long term phenotypic improvement in a large, outbred animal model. Though this pre-clinical context was encouraging, this and other experiments highlight the problem of unwanted immune responses to the therapeutic protein (FIG. 1). Moreover, it is well established clinically that over 30% of human patients with hemophilia A, i.e., deficiency of Factor VIII, develop inhibitory antibodies to recombinant Factor VIII protein infusions with subsequent loss of treatment efficacy. The following experiments were designed to test whether combine systemic gene transfer with a tolerogenic adoptive immune-modulatory strategy to suppress the resulting anti-Factor VIII immune response would result in long term expression of Factor VIII.

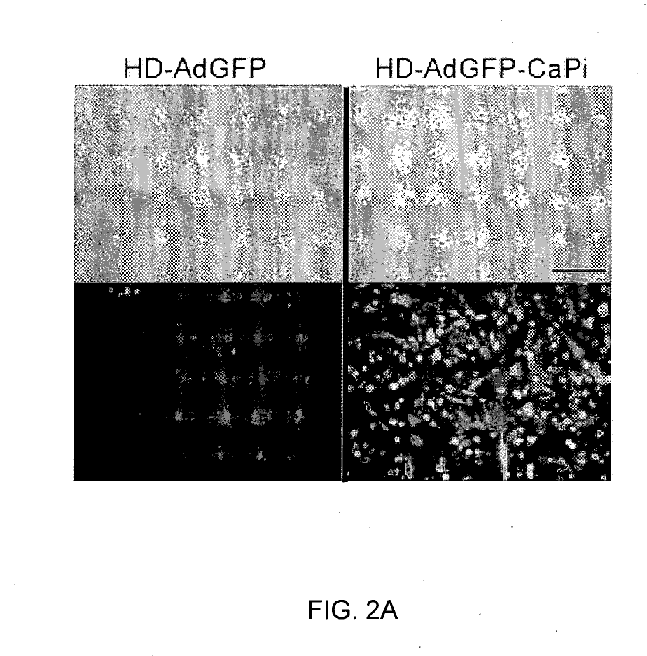

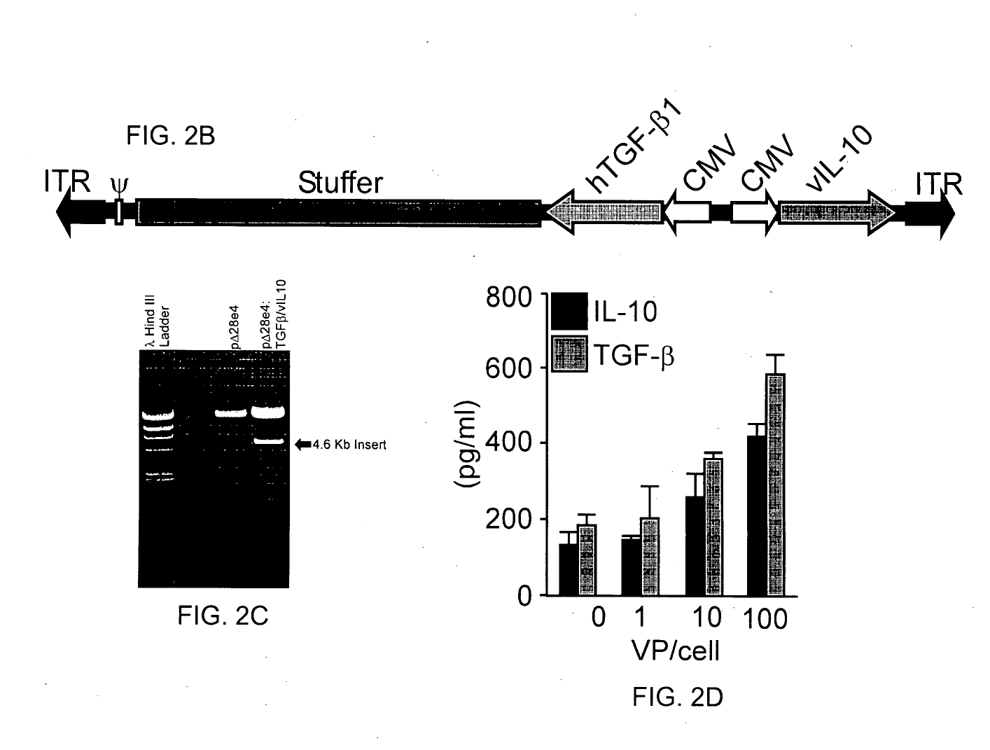

A helper-dependent adenovirus (HD-Ad) expressing the cytokines TGFβ and IL-10 (HD-Ad5TGF-β/IL-10 or otherwise referred as HDAd_{Tol}) was constructed (FIG. 2). Both molecules were previously shown to induce immunosuppressive and/or tolerogenic functions in both DCs and responding T cell populations.

To this end, bone marrow derived DCs were treated with 5000 vp/cell of either HD-AdGFP, or Hd-Ad complexed with calcium phosphate (HD-AdGFP:CaPi). After 1 hour, the vector was removed, and replaced with fresh media. Two days later GFP expression was imaged by live cell fluorescence microscopy (FIG. 2A). An apparent increase in GFP fluorescence of the HD-Ad:CaPi complex treated DCs was observed.

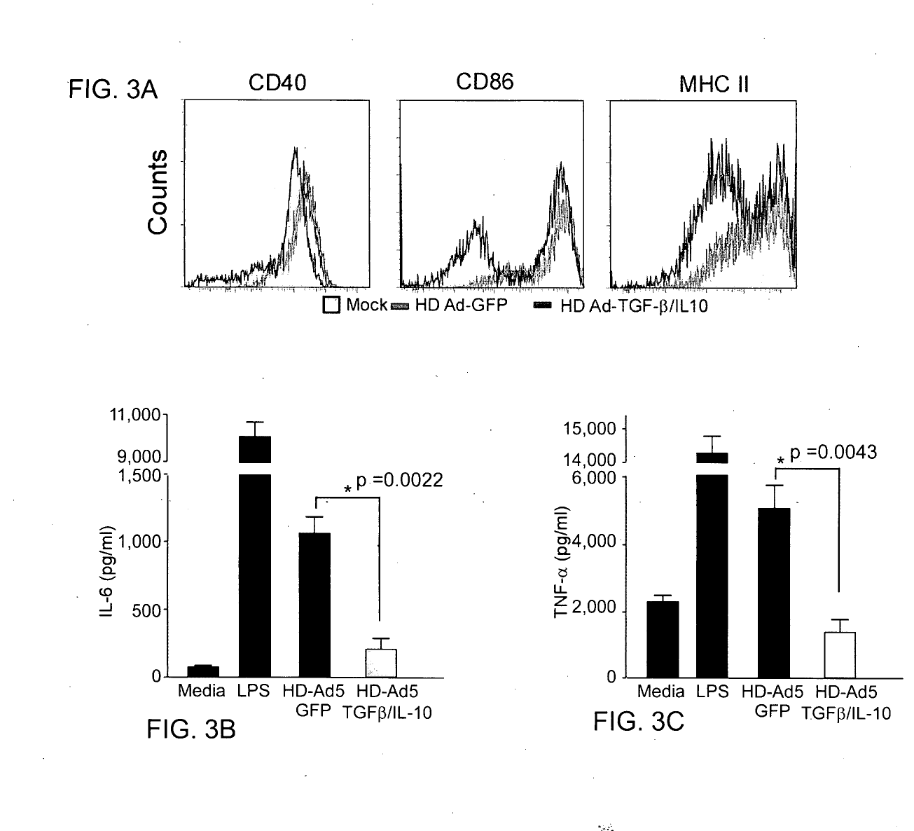

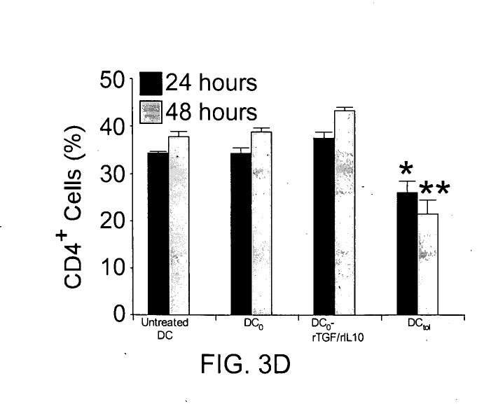

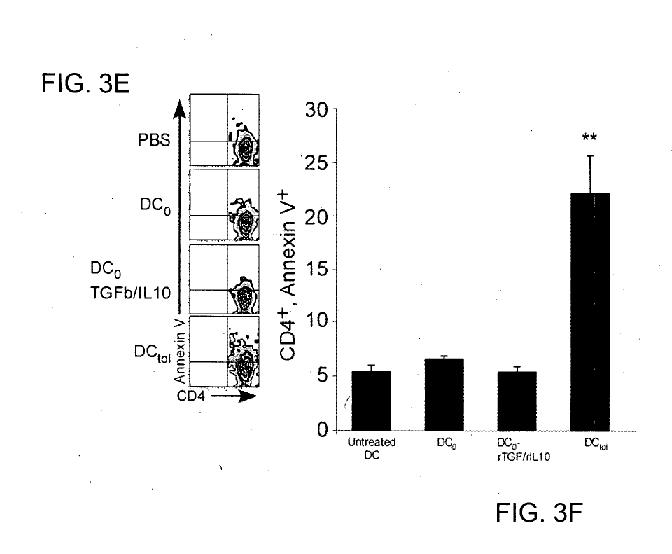

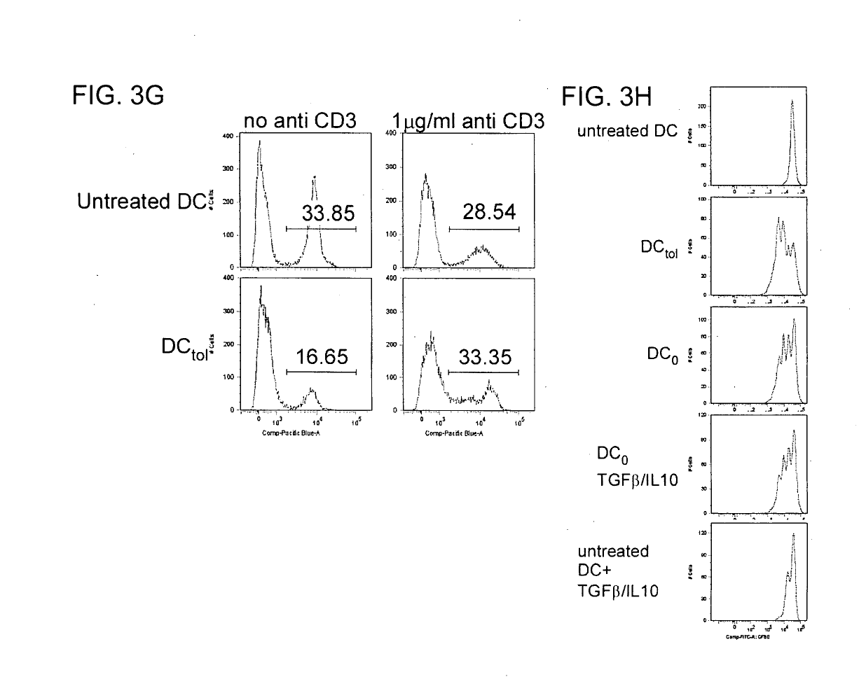

To genetically modify DC to promote immune tolerance, a HD-Ad (FIG. 2B) that simultaneously expressed human TGF-β1 and the Epstein-Barr virus encoded homologue of IL-10 (vIL-10) (HD-Ad5TGF-β/IL-10) was constructed (FIGS. 2C and 2D). In contrast to cellular IL-10, the vIL-10 homologue shows immunosuppressive properties but not stimulatory effects on NK cells and cytotoxic T lymphocytes (Ding et al., 2000 J Exp Med. 191: 213-224). Treating DC with HD-Ad5TGF-β/IL-10 did not significantly alter the typical induction of surface maturation markers compared to control HD-AdGFP treated DC, measured by levels of the costimulatory molecules CD40, CD86 and MHC II (FIG. 3A). Because surface maturation is only one component of functional DC activation, the secretion of pro-inflammatory cytokines from DC treated with each vector was compared. Media from DC alone, or DC treated with HD-Ad5GFP or HD-Ad5TGF-β/IL-10 was collected and the presence of IL-6 and TNF-α produced after 24 hours was measured. As positive control for cytokine secretion, DCs were incubated with the TLR agonist bacterial lipopolysaccharide (LPS) (1 μg/ml). As expected, the level of IL-6 increased 14-fold when vector alone was added to DC compared to mock treated DC (1068.07+/−125.05 pg/ml vs. −74.74+/−6.7 pg/ml) (FIG. 3B). However, DC treated with HD-Ad5TGF-β/IL-10 secreted nearly 5-fold less IL-6 compared to HD-Ad5GFP treated DC (211.54+/−80.81 pg/ml vs. 1068.07+/−125.05 pg/ml; p=0.002). Consistent with the reduction in IL-6, a 3.6-fold reduction in TNF-α secretion was observed (1364.60+/−393.91 pg/ml vs. 5039.92+/−666.25 pg/ml; p=0.004) (FIG. 3C). Interestingly, HD-AdGFP:CaPi treated DC had a markedly reduced induction of pro-inflammatory cytokines compared to LPS, suggesting that the activation effects after HD-Ad:CaPi transduction were relatively weak to begin with. Together these DC exhibited a mature-resting phenotype, thus resembling “tolerogenic” DC (Tan et al., J Leukoc Biol. 78: 319-324), and are referred elsewhere herein as DCₜₒₗ.

In summary, it was observed that HDAd_{Tol} did not alter DC expression of the maturation markers CD40 and CD86, while it did reduce secretion of TNFα and IL-6 (FIG. 3). Hence, DCs efficiently transduced (approximately 100%) using a modified Adenovirus Calcium Precipitation method (Seiler et. al Molecular Therapy 2006) was able to achieve expression of immunosuppressive cytokines with minimal maturation of the DC. The immunosuppressive phenotype was confirmed by suppression of autologous expression of inflammatory cytokines, i.e., TNFα and IL-6.

Since it was observed that HD-AdTGF-β/IL-0 attenuated pro-inflammatory cytokine secretion from the DC, the next set of experiments were designed to characterize the effect of DCₜₒₗ on responding T cells in vitro. Since the induction of apoptosis is one mechanism by which DC can induce T cell unresponsiveness and tolerance, DCₜₒₗ were tested to determine whether they exhibit apoptotic properties in vitro. Briefly, DCₜₒₗ from C57BL/6J mice were added to BALB/cJ splenocytes that were treated with activating anti-CD3 antibody in a robust allogeneic, one way mixed lymphocyte reaction. A significant reduced overall percentage of CD4⁺ cellularity was observed 24 and 48 hours after culture with DCₜₒₗ, but not with the control untreated DC (mock), DC treated with HD-Adzero, (a vector expressing no transgene (DC₀)), or DC₀ supplemented with exogenous recombinant TGF-β and IL-10 (FIG. 3D). It is believed that the decrease in responding CD4⁺T cells was explained by a substantial increase in CD4⁺ T cell apoptosis induced only in the cultures containing DCₜₒₗ (FIG. 3E-F). DC₀ added to the co-cultures in the presence of recombinant TGF-β and IL-10 was not sufficient to either reduce the frequency of CD4⁺ T cells, or increase the rate of apoptotic CD4⁺ T cells, suggesting cytokine secretion from the DC after transduction but prior to co-culture, or during T cell ligation is critical to the apoptotic stimuli. It is also believed that there is a dependence on the lack of T cell activation since fewer T cells were observed after 3 days in the DCₜₒₗ containing co-culture in the absence of anti CD-3 antibody than if anti-CD3 was added (FIG. 3G). Importantly, when anti-CD3 was added to the cultures, DCₜₒₗ did not prevent proliferation of the remaining T cells, suggesting the effects of DCₜₒₗ are not strictly apoptosis inducing (FIG. 3H).

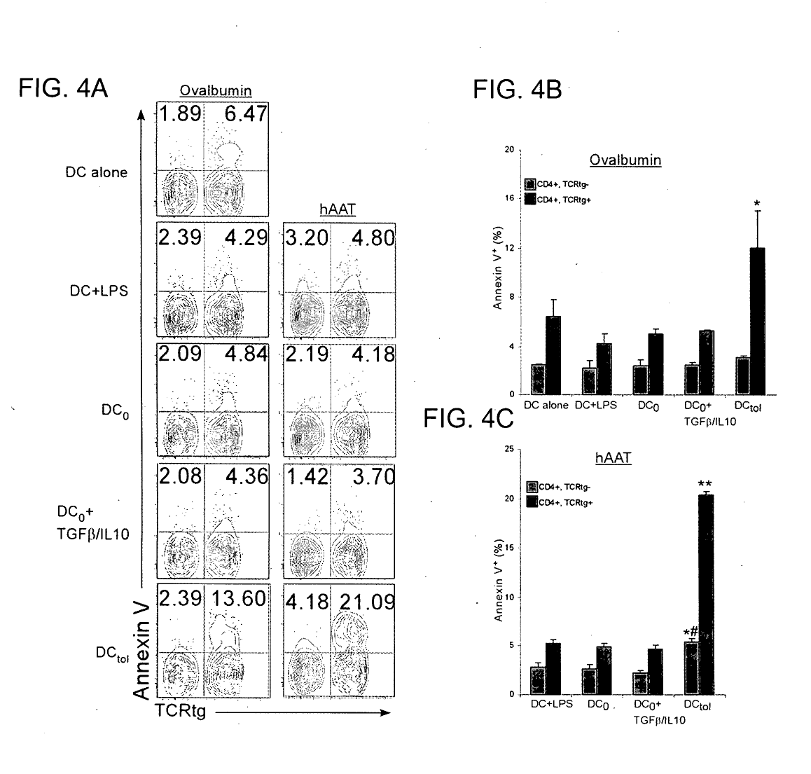

To investigate whether the induction of apoptosis by DCₜₒₗ was antigen specific, mixed lymphocyte reactions for the induction of T cell apoptosis was analyzed, this time using wild type BALB/cJ (H2^{d}) DCs in culture with syngeneic BALB/cJ (H2^{d}) splenocytes, and spiked with purified OVA-specific, (H2^{d})-restricted, D011.10 TCR tg T cells. In this model, the majority of T cells respond specifically to OVA and can be uniquely identified with antibodies to the transgenic TCR. Again, DCₜₒₗ were much more efficient at inducing apoptosis in responder T cells than control DC (FIG. 4A). Surprisingly however, DCₜₒₗ loaded with OVA were less efficient at inducing D011.10 T cell apoptosis than DCₜₒₗ cultured with an irrelevant antigen (hAAT), though significant increases were noted in both TCRtg⁺ and TCRtg⁻ T cells in culture with DCₜₒₗ-hAAT (FIG. 4B, 4C). Together, these data suggest DCₜₒₗ are capable of inducing apoptosis in reactive T cells, though they also have a strong effect on bystander T cells in the absence of TCR interaction in vitro.

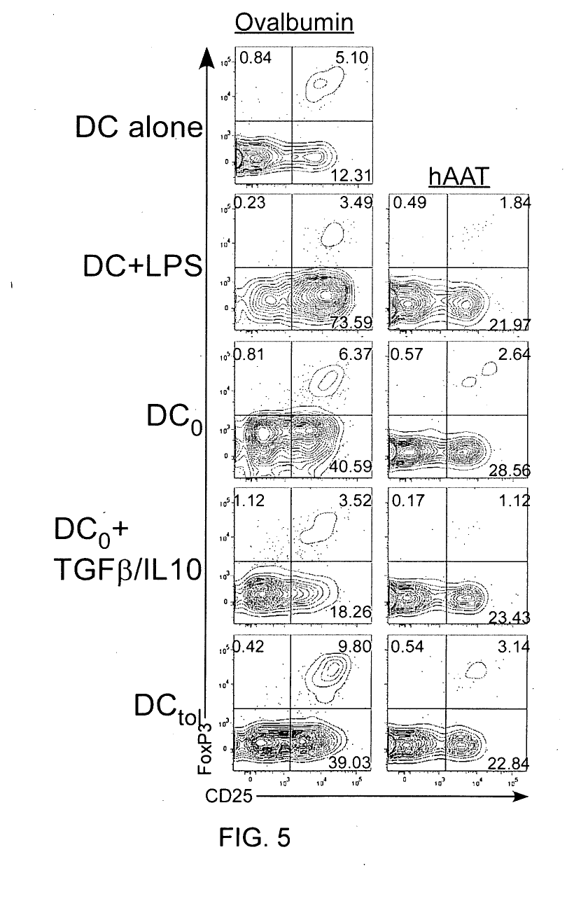

The induction of peripheral tolerance by DC occurs via apoptotic clearance of reactive T cells, as well as the active conversion or induction/expansion of Treg cells (Steinman et al., 2003 Annu Rev Immunol. 21: 685-711. To determine if this method for generating DCₜₒₗ supported the induction of antigen-specific Treg in vitro, mixed lymphocyte reactions were tested with D011.10 responder splenocytes cultured with varying conditions of DC in the presence either of OVA or of hAAT. Consistent with their tolerogenic effects in vivo, DCₜₒₗ-containing co-cultures included a higher frequency of Tregs (CD4⁺, CD25⁺, FoxP3⁺, TCR transgenic T cells) than DC₀, or DC₀ supplemented with recombinant TGF-β and IL-10, after 3 days in culture (FIG. 5). In contrast to the induction of T cell apoptosis however, Treg induction seemed to be antigen-specific because loading DC with irrelevant hAAT did not increase the frequency of Tregs. Taken together, these data suggest that the induction of T cell apoptosis, as well as favoring Treg generation and/or Treg survival are consistent with the functional ability to induce tolerance, at least in vitro.

The next set of experiments were designed to determine whether DCₜₒₗ could modulate immune responses in vivo. Syngeneic DCₜₒₗ loaded with either human albumin, or an irrelevant hAAT antigen were injected into recipient C3H/HeJ mice (FIG. 6A). Recipient mice were injected twice with the antigen-loaded DCₜₒₗ one week apart, the last occurring one week prior to immunization with albumin in complete freund's adjuvant (CFA). The total IgG anti-albumin immune responses were measured one month later. The adoptive transfer of DCₜₒₗ loaded with albumin protein suppressed the total anti-albumin IgG response in a majority of the recipient mice (6 of 10) compared to hAAT-loaded DCₜₒₗ (2 of 10) or mice receiving only immunization (0 of 9) (p=0.009) (FIG. 6B). Thus, in vitro and in vivo functions of DCₜₒₗ are consistent with the induction of immune tolerance. Moreover, antigen-loading was important in directing the response, suggesting antigen exposure shortly after vector transduction, and prior to injection is necessary to reduce antigen specific suppression of immunity.

The next set of experiments were designed to determine whether DCₜₒₗ prolongs FVIII gene therapy. Because helper-dependent FVIII gene therapy (and clinical FVIII protein therapy) is complicated by unwanted immune response to the transgene (Chuah et al., 2003 Blood 101: 1734-1743). The experiments were designed to test whether adoptively transferring DCₜₒₗ loaded with FVIII antigen would suppress the anti-FVIII humoral response following systemic gene transfer. DCₜₒₗ, DC treated with HD-Ad5GFP expressing GFP (DC_{HD-AdGFP}), or mock-treated DC harvested from FVIII-KO littermates were cultured with recombinant FVIII protein (4.7 IU/ml) immediately following HD-Ad:CaPi treatment, and 24 hours prior to adoptive transfer. As before, recipient mice were injected twice with the modified DC, one week apart, with the last occurring one week prior to systemic administration of HD-Ad5FVIII expressing human B domain deleted FVIII, or “gene therapy” (FIG. 7A). HD-Ad5FVIII gene therapy alone resulted in initial FVIII expression of 100% normal in all mice measured at one week post gene transfer, and was completely absent from plasma after week 3 (FIG. 7B). Recipient mice receiving mock-treated DC responded similarly, with 100% normal levels of FVIII expression at one week, but complete disappearance by week 3 post gene transfer. Levels of FVIII in mice receiving DC_{HD-AdGFP} peaked at one week post gene transfer, but decreased to undetectable levels in most mice by three weeks and in all mice at week 24. Adoptive transfer of DCₜₒₗ resulted in FVIII levels that peaked at 1 week in eight of eight mice which remained at levels in the therapeutic range between 8% and 100% for 24 weeks in 5 of the 8 mice. This experiment was repeated in two additional cohorts of animals with either a single DCₜₒₗ intervention or as described, with similar long-term persistence of FVIII activity.

The next set of experiments were designed to determine whether DCₜₒₗ mediated FVIII persistence is due to suppression of the antibody response. To understand the impact of DCₜₒₗ on the anti-FVIII immune response, total anti-FVIII IgG titers were measured twenty-four weeks after gene therapy (FIG. 8A). Consistent with the decrease in circulating FVIII, the anti-FVIII total IgG titer significantly increased in all of the animals receiving gene transfer alone, mock-treated DC, and most of the DC_{HD-AdGFP} treated mice. The adoptive transfer of DCₜₒₗ resulted in suppressed anti-FVIII antibody titers in six of eight treated mice (p=0.03; chi-square analysis). Together, these results indicate that the adoptive transfer of DCₜₒₗ inhibits the anti-FVIII immune response leading to long-term correction of FVIII deficiency.

To test the antigen-specificity of the immune suppression observed in FVIII-treated mice, we quantified their total neutralizing antibody responses to capsid proteins of the adenovirus vector. We reasoned that since the DCₜₒₗ were also challenged with adenoviral proteins from the HD-Ad5TGF-β/IL-10 vehicle, they could potentially suppress the anti-adenoviral immune response classically associated with the systemic vector treatment. However, when serum from each mouse obtained at week 24 post gene transfer was incubated with adenovirus in a neutralization assay, all conditions were found to equally respond with neutralizing antibodies against the vector (FIG. 8B). This indicates two important findings: First, the DCₜₒₗ was not sufficient to suppress the normal immune response to viral antigens after systemic vector administration. Second, loading DCₜₒₗ with FVIII protein was sufficient to drive FVIII specific immune suppression and thus prolong the efficacy of gene therapy. These data suggest that immune tolerance mediated by DCₜₒₗ can be antigen-specific in vivo and support the use of adoptive cell therapy as an adjunct treatment in gene and possibly protein therapy.

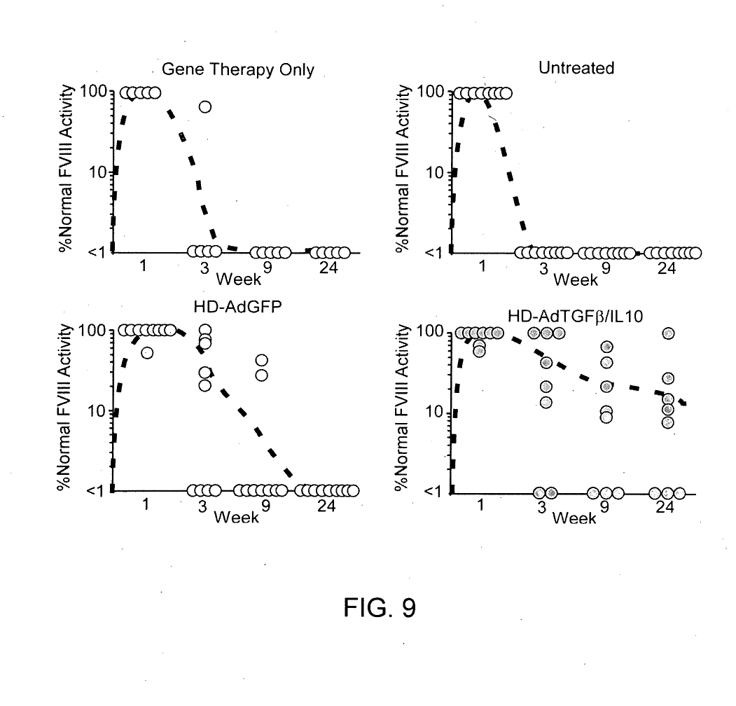

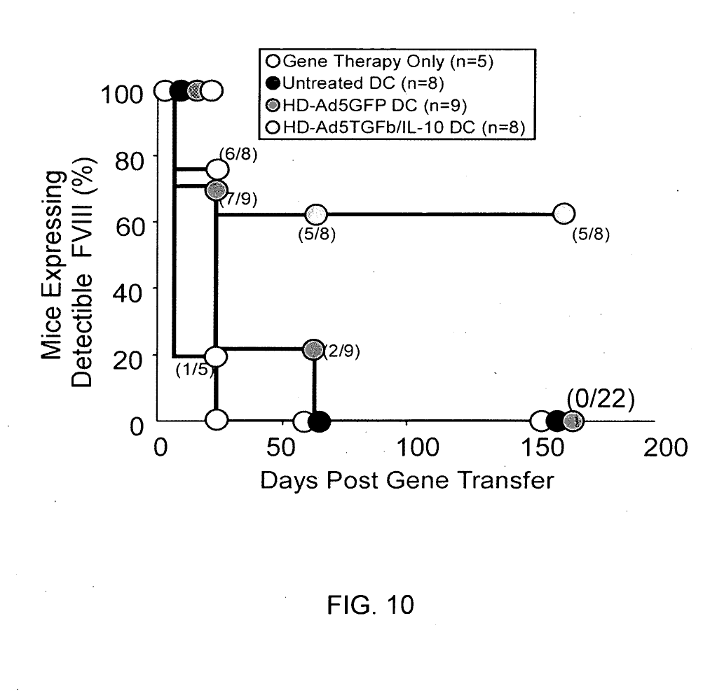

The next set of experiments were designed to determine whether DCs treated with HD-Adₜₒₗ would mediate targeted immune suppression in vivo. Factor VIII-loaded, HDAd_{Tol}-treated DCs were transferred into naïve Factor VIII knock-out mice. The recipient mice were then subjected to conventional systemic HD-Ad Factor VIII gene therapy. It was observed that adoptive DC transfer prolongs Factor VIII expression in the Factor VIII knock-out mice (FIG. 9). It was also observed that, five of eight mice injected with the HDAd_{Tol}-treated DC expressed levels of 10-100% normal Factor VIII for 24 weeks, whereas control mice lost all detectible Factor VIII expression by week 3 (FIG. 10). Moreover, the mice injected with the HDAd_{Tol}-treated DC suppressed the development of anti-FVIII antibodies; however this strategy was not sufficient to suppress the anti-adenovirus response. Hence, the biological activity of tolerogenic DCs were specific for the pulsed antigen, i.e., Factor VIII. It was also observed that HDAd_{Tol}-treated DC decreased the percentage of reactive CD4 T cells and increased T cell apoptosis.

In summary, DC modified with HDAd_{Tol} induced a tolerogenic-like phenotype, and after adoptive transfer, prolonged Factor VIII expression beyond that of adoptively transferred control DC.

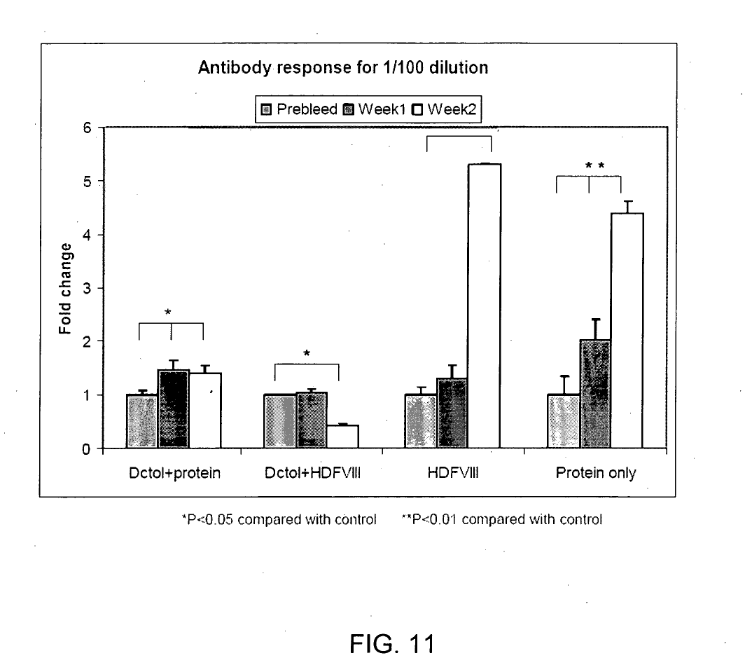

The next set of experiments were designed to determine whether DCs treated with HD-Adₜₒₗ would mediate targeted immune suppression to FVIII protein therapy in vivo. Briefly, FVIII deficient mice were pretreated with DCtol pulsed with FVIII. Treated vs. naïve mice then received either one control treatment of helper-dependent adenovirus expressing FVIII, or injection of recombinant human FVIIII at a dose of 0.3 IU/kg/dose every three days. Blood was sampled for measurement of antibody titers to human FVIII. As shown in FIG. 11, mice treated with DCtol suppressed an immune response to FVIII irrespective of whether FVIII was produced by gene transfer of repeated FVIII infusions. Control mice treated with the FVIII gene therapy or protein therapy both expressed a robust antibody response by two weeks post initial treatment. These data support the applicability of this approach to suppression of antibody response to protein therapies. In this study, it was observed that long-term Factor VIII gene expression is related to suppression of the anti-Factor VIII antibody response in adult FVIII knockout mice. Taken together, these data demonstrate that using helper-dependent Ad mediated gene transfer to express immuno-modulatory molecules in this adoptive transfer strategy can confer tolerance to endogenously produced or exogenously delivered antigens.

The results presented herein demonstrate that modifying DCs to enhance their tolerogenic potential is useful in improving long term gene expression. This strategy can be applicable to gene therapy for any situation where a potential neo-antigen is either expressed or directly delivered.

### Example 2

### Dendritic Cell Therapy for Tolerance Induction

FVIII specific inhibitor formation in both mice and humans is a CD4⁺ T cell dependent mechanism requiring T cell interaction with DC and B cells (Lacroix-Desmazes et al., 2002 Autoimmun Rev 1: 105-110; Wu et al., 2001 Thromb Haemost 85: 125-133). Since DCs are key regulators of downstream T cell responses, they are an attractive target to re-program antigen presentation and harness the resulting immune response. The results presented herein demonstrate a new method of enhancing FVIII gene transfer by at least regulating the immune response directed against FVIII. In the present study, FVIII was used as a non-limited example for the strategy of targeted immune suppression as adjunct prophylaxis to prolong the duration of FVIII gene therapy.

HD-Ad was engineered to express the immuno-modulatory cytokines TFGβ and IL-10 at a sufficient level to attenuate DC activation, induce apoptosis, and increase the frequency of antigen-specific Treg cells in vitro. The induction of apoptosis was previously shown to be important for experimental tolerance to FIX in mice (Mingozzi et al., 2003 J Clin Invest. 111: 1347-1356). The in vitro results presented herein demonstrated that DCₜₒₗ induced substantial apoptosis in both bystander CD4⁺ T cells and in antigen-specific T cells. Despite increased apoptosis in responder CD4⁺ T cells, the frequency of CD4⁺, CD25⁺, FoxP3⁺, TCRtg T cells increased, suggesting that DCₜₒₗ were not inducing clearance of this T cell subset, and could support their differentiation. These aspects of DCₜₒₗ function are consistent with tolerance induction. More importantly, stable levels of FVIII ranging from 8% to 100% normal were maintained for six months in mice pre-treated with DCₜₒₗ; and this was attributed to suppression of anti-FVIII immunity.

Since induction of Treg cells in vitro and overall immune suppression in vivo in the present study appeared to be antigen-specific with DCₜₒₗ, this methodology avoids the unwanted complication of general immune suppression. Thus, ex vivo genetic manipulation of antigen-loaded DC affords the opportunity not only to specify the tolerizing antigen, but also to achieve a tolerogenic response to therapeutic proteins. Without wishing to be bound by any particular theory, the methods discussed herein can be applied to strategies of combined cell and optimized systemic gene transfer and demonstrate the feasibility of gene replacement therapy. It is believed that gene replacement therapy is a more likely target for successful translation of cell-mediated immune modulation over the use of solid organ transplantation, given the singularity of the neo-antigen. Hence, this study emerges as the first report of a clinically-relevant autologous cell therapy to achieve targeted immune suppression in adult animals.

In summary, the implications of this report span multiple disease modalities, from the hemophilia A model of monogenic disease gene therapy described here, to other clinical manifestations of anticipated, unwanted immune responses. Examples include the induction of anti-drug antibodies resulting from repeated protein therapies, as well as solid organ transplantation, and auto-immune diseases like diabetes, and inflammatory bowel disease.

### Example 3

### Dendritic Cell Mediated Adoptive Immune-Modulation Suppresses the Antibody Response to CFA/Albumin

The following experiments were designed to test whether combine systemic gene transfer with a tolerogenic adoptive immune-modulatory strategy to suppress the immune response in an antigen specific manner. In the Example, the antigen of interest is albumin.

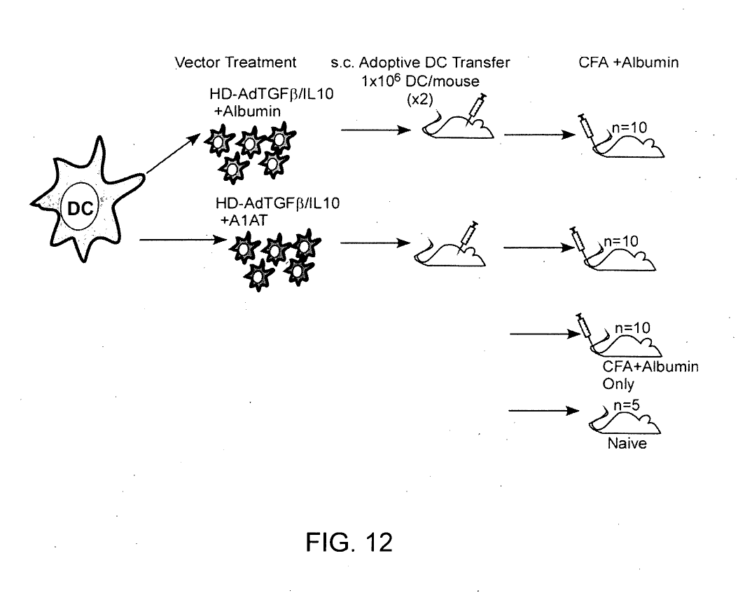

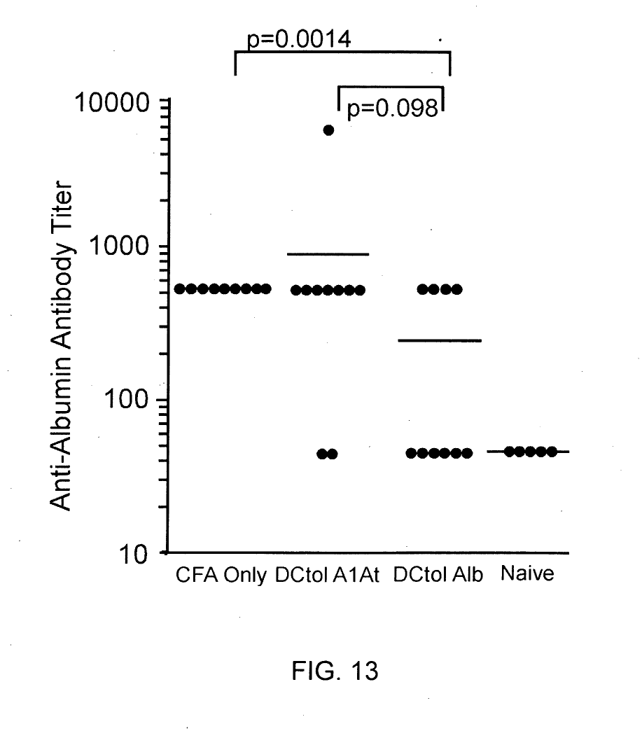

The next set of experiments were designed to determine whether DCs treated with HD-Adₜₒₗ would mediate targeted immune suppression in vivo. Albumin-loaded, HDAd_{Tol}-treated DCs were transferred into naïve out mice (Alpha 1 antitrypsin-loaded, HDAd_{Tol}-treated DCs were transferred into naïve mice as a control). The recipient mice were then subjected to immunogenic challenge with Complete Freunds Adjuvant (CFA) and albumin (FIG. 12). It was observed that adoptive DC transfer suppressed the development of anti-albumin antibody titer (FIG. 13).

The results presented herein demonstrate that DCs engineered with a helper-dependent adenovirus (HD-Ad) expressing the cytokines TGFβ and IL-10 (HDAd_{Tol}) can suppress an antibody response in an antigen specific fashion.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A dendritic cell genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof.

2. The dendritic cell of claim 1, further comprising an antigen having at least one epitope.

3. The dendritic cell of claim 2, wherein said antigen is expressed in said cell by an expression vector.

4. The dendritic cell of claim 2, wherein said antigen is delivered directly to said cell as a pulse of a protein.

5. The dendritic cell of claim 2, wherein said antigen is delivered directly to said cell as a mixture of proteins that are purified or are from cell/tissue lysates.

6. The dendritic cell of claim 2, wherein said antigen is associated with a disease or a therapeutic treatment.

7. The dendritic cell of claim 6, wherein said disease is selected from the group consisting of an infectious disease, a cancer and an autoimmune disease.

8. A method of inducing immune tolerance in a mammal, the method comprising administering a dendritic cell genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof.

9. The method of claim 8, wherein said dendritic cell further comprises an antigen having at least one epitope.

10. The method of claim 9, wherein said antigen is expressed in said cell by an expression vector.

11. The method of claim 9, wherein said antigen is delivered directly to said cell as a pulse of a protein.

12. The method of claim 9, wherein said antigen is delivered directly to said cell as a mixture of proteins that are purified or are from cell/tissue lysates.

13. The method of claim 9, wherein said antigen is associated with a disease or a therapeutic treatment.

14. The method of claim 9, wherein said disease is selected from the group consisting of an infectious disease, a cancer and an autoimmune disease.

15. A method of treating a transplant recipient to reduce in said recipient an immune response against the transplant, the method comprising administering to a transplant recipient, a dendritic cell genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof, in an amount effective to reduce an immune response against the transplant.

16. The method of claim 15, wherein said transplant is selected from the group consisting of a biocompatible lattice, a donor tissue, an organ, a cell, a nucleic acid, a protein, and any combination thereof.

17. The method of claim 15, wherein said dendritic cell further comprises an antigen having at least one epitope, wherein said antigen is associated with the transplant.

18. The method of claim 17, wherein said antigen is expressed in said cell by an expression vector.

19. The method of claim 17, wherein said antigen is delivered directly to said cell as a pulse of a protein.

20. The method of claim 17, wherein said antigen is delivered directly to said cell as a mixture of proteins that are purified or are from cell/tissue lysates.

21. The method of claim 17, wherein said dendritic cell is administered to the transplant recipient to treat rejection of the transplant by the recipient.

22. The method of claim 15, further comprising administering to said recipient an immunosuppressive agent.

23. The method of claim 15, wherein said dendritic cell is administered to the recipient prior to said transplant.

24. The method of claim 15, wherein said dendritic cell is administered to the recipient concurrently with said transplant.

25. The method of claim 15, wherein said dendritic cell is administered simultaneously with said transplant.

26. The method of claim 15, wherein said dendritic cell is administered to the recipient subsequent to the transplantation of said transplant.

27. A method of enhancing the expression of a protein in a mammal, the method comprising administering a dendritic cell genetically modified to express at least two immunosuppressive molecules selected from the group consisting of interleukin 2 (IL-2), interleukin 4 (IL-4), interleukin-6 (IL-6), interleukin 10 (IL-10), interferon γ, macrophage migration inhibitory factor (MIF), lymphotoxin β (LTB), transforming growth factor β (TGFβ), and any combination thereof, into said mammal thereby enhancing expression of said protein.

28. The method of claim 27, wherein said DC further comprises an antigen having at least one epitope.

29. The method of claim 28, wherein said antigen is expressed in said cell by an expression vector.

30. The method of claim 28, wherein said antigen is delivered directly to said cell as a pulse of a protein.

31. The method of claim 28, wherein said antigen is delivered directly to said cell as a mixture of proteins that are purified or are from cell/tissue lysates.

32. The method of claim 28, wherein said antigen is associated with said protein.

33. The method of claim 27, wherein said protein is expressed in said mammal as a result of gene therapy.

34. The method of claim 27, wherein said protein is a therapeutic protein.

35. The method of claim 27, wherein said protein is selected from the group consisting of a hormone, monoclonal antibody, an enzyme, a cytokine, a toxin, a fusion protein, and any combination thereof.

36. The method of claim 27, wherein said protein is selected from the group consisting of FVIII, insulin, thrombopoietin (TPO), erythropoietin (EPO), interferon-β (INF-β), INF-α, GM-CSF, tissue plasminogen activator, myelin basic protein (MBP), AXO, and any combination thereof.
